(19) United States
(12) Patent Application Publication
(10) Pub. No.: US 2025/0081293 A1
(43) Pub. Date: Mar. 6, 2025

(54) WEARABLE THERMAL SENSORS, SYSTEMS AND METHODS THEREOF

(71) Applicant: RHAEOS, INC., Evanston, IL (US)

(72) Inventors: Richard Chad WEBB, St. Paul, MN (US); Adam ZYSK, Chicago, IL (US); Shannon MCKAY, Evanston, IL (US); Blake PARSONS, Chicago, IL (US); Scott ODLAND, Chicago, IL (US)

(21) Appl. No.: 18/725,949

(22) PCT Filed: Dec. 30, 2022
(86) PCT No.: PCT/US2022/082608

§ 371 Date: Jul. 1, 2024

Related U.S. Application Data
(60) Provisional application No. 63/295,193, filed on Dec. 30, 2021.

(51) Int. Cl. H05B 1/02; A61M 27/00; G01F 1/684; G01F 1/699; G01K 13/20

(52) U.S. Cl. CPC H05B 1/025; A61M 27/006; G01F 1/6847; G01F 1/699; G01K 13/20

(57) ABSTRACT

Systems and methods for monitoring temperature using wireless, flexible thermal sensors are disclosed. A wireless, flexible thermal sensor mountable on a body may comprise a substrate, a power source, a thermal actuator configured to receive power from a power source and supply thermal energy to a portion of a skin surface of the body, a temperature sensor configured to detect a change in a temperature related to the thermal actuator, and a comparator configured to receive an electrical signal corresponding to the temperature of the temperature sensor, compare the received signal to a reference signal, and output a signal based on the comparison. The sensor may further comprise a power control element configured to receive a signal corresponding to the output signal of the comparator and alter the delivery of power to the thermal actuator based at least in part on the signal received from the comparator.

### CROSS REFERENCE TO RELATED APPLICATION(S)

This application claims priority to International Application No. PCT/US2022/082608, filed on Dec. 30, 2022, which claims priority to U.S. Provisional Application No. 63/295,193, filed Dec. 30, 2021, the contents of these applications are incorporated herein by reference in their entireties.

### TECHNICAL FIELD

The description herein generally relates to the field of cerebrospinal fluid shunts, and more particularly to systems and methods of monitoring flow of cerebrospinal fluid in shunts using non-invasive, wearable epidermal electronics.

This invention was made with government support under NSF Award 1938472 awarded by National Science Foundation (NSF). The government has certain rights in the invention.

### BACKGROUND

Thermal sensors are used to monitor the temperature of objects and, when utilizing a thermal sensor which has an electrical property which varies in response to temperature change, can be implemented as part of an electrical sensing system. A sensor for monitoring thermal transport properties of an object can be created by combining one or more temperature sensors with one or more thermal actuators as part of a sensing system. By measuring the temperature change of an object in response to actuation by the thermal actuator, various thermal transport properties can be determined, such as thermal conductivity, heat capacity, diffusivity, and other related parameters. Different spatial locations of temperature sensors relative to the thermal actuator result in different thermal responses to thermal actuation. Some arrangements of temperature sensors and thermal actuators additionally enable measurement of convective thermal transport properties induced by the movement of fluids near to the thermal actuator. While fluid flow sensors based on thermal actuation, such as hot-wire anemometers, are often desired to be in direct contact with a fluid, certain sensor arrangements enable fluid flow measurements through a solid media, without direct contact with the fluid. For example, such designs can enable body-mountable sensors for noninvasive measurement of biological fluid flow beneath the skin surface without directly contacting the biological fluid.

There are several challenges associated with making skin-mountable thermal sensors, and in particular, noninvasive thermal fluid flow sensors which are precise, accurate, and reliable. One of such challenges is minimizing the thermal resistance between actuators and/or sensors and the measurement object, which may be a bodily fluid beneath the skin. Some of the currently existing conventional designs employ materials between the sensors/actuators and the skin which create thermal resistance, thereby limiting the accuracy and reliability of the measurements. Some existing sensors are mechanically rigid, which limits their ability to intimately conform to a skin surface without high resistance air gaps. Furthermore, conventional electronic constructions may create a low thermal resistance between thermal actuators and multiple temperature sensors, thereby limiting relative signal response to the object of interest. Additional challenges may also exist due to influence of undesirable external factors, such as air flow over the sensors, which may reduce the signal reliability of body tissues below the skin. In the case of thermal fluid flow sensors, a specific orientation of the sensor relative to the underlying conduit may provide improved signal levels. Some mechanical designs may optimize usability of the sensor for alignment to an underlying conduit. Embodiments described herein serve to address one or more of these challenges by employing multiple constructions to optimize signals by modifying the mechanical designs. Furthermore, wearable thermal sensors require a combination of intimate thermal sensor contact and comfortable wearability from a combination of low mass and comfortable form factor. Currently existing designs may be unable to achieve each parameter simultaneously due to either large mass or rigid and uncomfortable form factor. Embodiments disclosed herein describe constructions to resolve each issue simultaneously.

In addition to the above-mentioned challenges, other challenges related to electrical signals may exist with skin-mountable thermal sensors. Thermal actuators applied to tissue, such as skin, may represent a hazard to the user if the thermal actuation creates excess heat. Conventional skin-mounted thermal actuators do not account for possible tissue variations in constant power systems, or possible component failure in constant temperature systems, or do not account for expected component variability in manufacturing. Embodiments disclosed herein describe constructions for creating hardware-controlled, software-controlled, and redundant thermal failsafe systems. Further, conventional skin-mounted thermal actuation sensors require large batteries, suffer short shelf-life, or require mechanical openings to charge or replace batteries, which may act as ingress points for damage, safety issues, or signal perturbations. Embodiments disclosed herein enable solutions for compact, long shelf-life, and hermetically scaled designs.

Numerous examples of applications for skin-mounted noninvasive thermal fluid flow sensors exist due to the multitude of fluid systems in the body. Exemplary fluid systems measurable using sensors may include, but are not limited to, blood, lymphatic fluid, and spinal fluid. As a non-limiting example, Hydrocephalus, represents an exemplary condition for spinal fluid flow measurements. Hydrocephalus is a common and costly condition caused by the accumulation of cerebrospinal fluid (CSF) in the brain, with symptoms that include headaches, seizures, coma, or death. It particularly afflicts children, occurring in 1-5 of every 1,000 live births. Hydrocephalus is usually treated with the surgical implantation of a catheter, known as a ventricular shunt, which diverts the excess CSF in the brain to a distal absorptive site, such as the peritoneal cavity. Overall, 40,000 shunts are implanted or replaced annually in United States, with costs estimated at $2 billion per year. Unfortunately, shunts have extremely high failure rates due to a diverse set of factors including occlusion, mispositioning, or kinking. Up to 51% of shunt recipients exhibit symptoms that require a shunt repair surgery, known as a “revision,” in the first year and 99% of shunts are replaced at least once within 10 years. It is also estimated that nearly half of all healthcare expenditures related to shunt surgery are spent on revisions. Non-specific symptoms like headaches and nausea make diagnosing shunt malfunction extremely challenging. Consequently, physicians rely on a complicated, expensive, and often inconclusive suite of tests, including those that expose patients to significant radiation, to make clinical decisions.

On average, a single pediatric hydrocephalus patient with an implanted shunt is admitted to the hospital multiple times each year, with a high number of emergency department presentations. The standard-of-care methods used to assess shunt malfunction today include (i) aspiratory CSF sampling, an invasive procedure used to assess pressure in the shunt, (ii) injection of a radionucleotide tracer into the ventricular system and monitoring the movement of the radiotracer into the abdomen, (iii) an x-ray “shunt series” exam to check for mechanical shunt damage, and (iv) Computerized Tomography (CT) or Magnetic Resonance Imaging (MRI) to assess ventricle size. Notably, CT and x-ray shunt series expose the patient to significant radiation. Also, the only test that directly measures CSF flow in the shunt, the radiotracer test, is invasive, painful, and carries a risk of infection. Moreover, the diagnostic performance of these tests is poor. X-ray shunt series, for example, has a sensitivity of approximately 18% for the determination of shunt failure in symptomatic patients. Finally, MRI and CT have poor performance because shunt failure is often not concomitant to ventriculomegaly. These tests are regularly employed despite their serious limitations because they are, unfortunately, the best tools currently available to clinicians.

In addition to their poor performance, frequent CTs expose children to sizeable radiation doses, increasing their risk of brain cancer. Although MRI is seen as a viable alternative to CT for evaluation of hydrocephalus, access and cost are significant barriers. Significantly, only ˜17% of the CTs performed lead to a revision surgery within a week, implying that the vast majority of these scans are unnecessary.

Therefore, the current standard of care, ventricular catheters (shunts), is prone to failure, which can result in nonspecific symptoms such as headaches, dizziness, and nausea. Current diagnostic tools for shunt failure such as CT, MRI, radionuclide shunt patency studies (RSPSs), and ice pack-mediated thermodilution have disadvantages including high cost, poor accuracy, inconvenience, and safety concerns. As an example, ShuntCheck® utilizes an ice-pack based thermal cooling system connected to a Windows PC DAQ to address a need for shunt monitoring. That technology, however, is cumbersome and time-consuming. The device's cumbersome, multi-step protocol; equivocal or negative past clinical studies; and need for ice-pack cooling have limited its acceptance. Additionally, patient discomfort due to prolonged skin cooling (detrimental for pediatric diagnostics) and absence of chronic monitoring further limits its diagnostic relevance. Accordingly, there is a need for a precise, rapid, easy-to-use, wireless, non-invasive shunt diagnostic, that is conformable to the skin and has epidermal-like mechanical properties.

Furthermore, other desirable features and characteristics will become apparent from the subsequent detailed description and the appended claims, taken in conjunction with the accompanying drawings and this background of the disclosure.

### SUMMARY

Embodiments of the present disclosure provide systems and methods of monitoring flow of cerebrospinal fluid through shunts. Disclosed herein are exemplary systems and methods of measurements of flow follow from localized thermal actuation and sensing using a flexible, non-invasive device gently laminated onto the surface of the skin at the location of the shunt. The results presented here extend these concepts into a user-friendly, fully wireless system that enables continuous, noninvasive monitoring of CSF flow performed by patients themselves in real-world settings. Advanced designs and integration schemes exploit low-cost commercial components and flexible circuit board manufacturing techniques in optimized layouts guided by theoretical and numerical models of thermal transport and system-level mechanics. A Bluetooth Low-Energy System on a Chip (BLE-SoC) embedded system architecture allows for robust, high-quality data transfer during normal patient activities, where a miniaturized on-board, rechargeable battery supports continuous operation for several hours. On-body measurements and field trials on hydrocephalus patients reveal reliable operation during both short ‘spot-checks’ and, for the first time, extended measurements of flow during natural motions of the body and for different orientations. The results suggest broad applicability for monitoring of shunts in patients across age ranges, pathologies, and settings, including the home.

In one aspect of the disclosure, a wireless, flexible thermal sensor mountable on a body is disclosed. The sensor may comprise a substrate, a power source, a thermal actuator supported by the substrate and configured to receive power from a power source and supply thermal energy to a portion of a skin surface of the body. The sensor may further include a temperature sensor supported by the substrate and configured to detect a change in a temperature related to the thermal actuator, a comparator supported by the substrate and configured to receive an electrical signal corresponding to the temperature of the temperature sensor, compare the received signal to a reference signal, and output a signal based on the comparison. The sensor may further include a power control element supported by the substrate and configured to receive a signal corresponding to the output signal of the comparator; and alter the delivery of power to the thermal actuator based at least in part on the signal received from the comparator.

In another aspect of the disclosure, a wireless, flexible thermal sensor mountable on a body is disclosed. The sensor may comprise a substrate, a thermal sensor supported by the substrate, a microprocessor supported by the substrate, a first region comprising the region of the substrate supporting the thermal sensor, a second region comprising the region of the substrate supporting the microprocessor, and an adhesive structure. The adhesive structure may comprise a first face configured for bonding to skin, and a second face opposite the first face. The second face may comprise an adhesive region bonded to the first region of the substrate and a non-adhesive region.

In yet another aspect of the disclosure, a wireless, flexible thermal flow sensor mountable on a body is disclosed. The sensor may comprise a substrate, a power source, a thermal actuator supported by the substrate and configured to receive power from a power source and supply thermal energy to a portion of a skin surface of the body, a temperature sensor supported by the substrate and configured to detect a change in a temperature related to the thermal actuator and an enclosure comprising a plurality of indicators visible on an external surface; wherein at least two indicators are located on opposite sides of the thermal actuator.

In yet another aspect of the disclosure, a wireless, flexible thermal flow sensor mountable on a body is disclosed. The sensor may comprise a substrate, a power source, a thermal actuator supported by the substrate and configured to receive power from a power source and supply thermal energy to a portion of a skin surface of the body, a temperature sensor supported by the substrate and configured to detect a change in a temperature related to the thermal actuator and an enclosure bonded to the substrate, wherein the external surfaces of the enclosure and the substrate form an unbroken seal around the sensor.

In yet another aspect of the disclosure, a computer-implemented system for monitoring a subdermal fluid conduit is disclosed. The system may comprise a wireless subdermal fluid flow sensor configured for attachment to skin, and a sensor control device in wireless communication with the fluid flow sensor. The sensor control device may include a graphical display, a memory storing instructions, and a processor configured to execute the instructions to display a set of instructions to a user to place the fluid flow sensor over the subdermal fluid conduit, receive date from the sensor, and display a result at least partially based on the received data on the graphical display.

In yet another aspect of the disclosure, a thermal flow-sensor mountable on a body is disclosed. The thermal sensor comprises a substrate, a power source, a thermal actuator supported by the substrate and configured to receive power from the power source and supply thermal energy to a portion of a skin surface of the body, a first temperature sensor supported by the substrate and configured to detect a change in a temperature of the thermal actuator, a second temperature sensor supported by the substrate and configured to detect a change in a temperature associated with a bodily fluid under the skin surface, and a plurality of current-regulating mechanisms configured to regulate electrical power to the thermal actuator based on the change in the temperature.

It will be understood that the foregoing description and the following detailed description are exemplary and explanatory only, and are not restrictive of the disclosed embodiments.

### BRIEF DESCRIPTION OF THE DRAWINGS

FIG. 1 is a schematic illustration of an exemplary wireless flow sensor including different device regions, consistent with disclosed embodiments.

FIG. 2 is a schematic illustration of an exemplary wireless flow sensor including patterned electrically conductive layers, consistent with disclosed embodiments.

FIG. 3 is a schematic illustration of an exemplary wireless flow sensor including a molded enclosure with regions of differing height, consistent with disclosed embodiments.

FIG. 4 is a schematic illustration of an exemplary wireless flow sensor including an elastomer enclosure with regions of differing height, consistent with disclosed embodiments.

FIG. 5 is a schematic illustration of an exemplary wireless flow sensor including an elastomer enclosure enclosing electronic components, consistent with disclosed embodiments.

FIG. 6 is a schematic illustration of an exemplary layered structure of an exemplary wireless flow sensor including an elastomer enclosure enclosing electronic component, consistent with disclosed embodiments.

FIG. 7 is a schematic illustration of an exemplary wireless flow sensor including an adhesive skirt, consistent with disclosed embodiments.

FIG. 8 is a schematic illustration of an exemplary wireless flow sensor including regions with direct bonding to skin contacting adhesives and regions with indirect bonding to skin contacting adhesives, consistent with disclosed embodiments.

FIG. 9 is a schematic illustration of an exemplary wireless flow sensor including an insulating media disposed on thermal sensors and actuators, consistent with disclosed embodiments.

FIGS. 10A and 10B are schematic illustrations of exemplary layered structures of a wireless flow sensor, consistent with disclosed embodiments.

FIG. 11 is a schematic illustration of an exemplary wireless flow sensor including a patterned surface indicating a line corresponding to an alignment axis of sensors, consistent with disclosed embodiments.

FIG. 12 is a schematic illustration of an exemplary wireless flow sensor including a shaped edge indicating a line corresponding to an alignment axis of sensors, consistent with disclosed embodiments.

FIGS. 13A and 13B are schematic illustrations of an exemplary wireless flow sensor including a magnetic switch and a corresponding exemplary circuit, respectively, consistent with disclosed embodiments.

FIG. 14 is a schematic illustration of an exemplary packaging comprising a magnet for a wireless flow sensor including a magnetic switch, consistent with disclosed embodiments.

FIG. 15 is a schematic illustration of an exemplary thermal actuator including a temperature sensor located within the thermal actuator, consistent with disclosed embodiments.

FIG. 16A is a schematic illustration of an exemplary comparator circuit for a thermal sensor, consistent with disclosed embodiments.

FIG. 16B is a schematic illustration of an exemplary device configuration including a thermal sensor, consistent with disclosed embodiments.

FIG. 17A is a schematic illustration of an exemplary thermal control circuit for a thermal actuator, including a comparator circuit, consistent with disclosed embodiments.

FIG. 17B is a schematic illustration of an exemplary device configuration including a thermal sensor, consistent with disclosed embodiments.

FIG. 18 is a schematic illustration of an exemplary thermal control circuit for a thermal actuator, including a redundant control mechanism, consistent with disclosed embodiments.

FIG. 19 is a schematic illustration of an exemplary system for wireless control of a thermal flow sensor via a sensor control device, consistent with disclosed embodiments.

FIG. 20 is a set of exemplary graphical display images to be displayed on a sensor control device for guiding a user on use of a thermal flow sensor, consistent with disclosed embodiments.

FIG. 21 is an optical image of an exemplary thermal flow sensor comprising a plurality of physical components for attachment to skin, consistent with disclosed embodiments.

FIG. 22 is a schematic illustration of an exemplary system including a wireless flow sensor, a sensor control device, and further computing devices and datastores, consistent with disclosed embodiments.

FIG. 23 is a schematic illustration of an exemplary system including a wireless flow sensor, a sensor control device, and further computing devices and datastores, consistent with disclosed embodiments.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following description of exemplary embodiments do not represent all implementations consistent with the disclosure. Instead, they are merely examples of apparatuses and methods consistent with aspects related to the subject matter recited in the appended claims. The following detailed description refers to the accompanying drawings. While several illustrative embodiments are described herein, modifications, adaptations and other implementations are possible. For example, substitutions, additions, or modifications may be made to the components and steps illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope of the invention is defined by the appended claims.

Relative dimensions of components in drawings may be exaggerated for clarity. Within the following description of drawings, the same or like reference numbers refer to the same or like components or entities, and only the differences with respect to the individual embodiments are described.

As used herein, unless specifically stated otherwise, the term “or” encompasses all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

In general, the terms and phrases used herein have their art-recognized meaning, which can be found by reference to standard texts, journal references and contexts known to those skilled in the art. The following definitions are provided to clarify their specific use in the context of the invention.

“Soft” refers to a material that may be comfortably positioned against the skin without discomfort or irritation to the underlying skin by the material itself deforming to conform to the skin without unduly exerting force on the underlying skin with corresponding device-generated skin deformation. Softness/hardness may be optionally quantified, such as in terms of durometer, or a material's resistance to deformation. For example, the substrate may be characterized in terms of a Shore 00 hardness scale, such as a Shore 00 that is less than 80. Soft may also be characterized in terms of a modulus, such as a Young's modulus that is less than or equal to 100 kPa.

“Stretchable” refers to a material's ability to undergo reversible deformation under an applied strain. This may be characterized by a Young's modulus, a ratio of stress to strain. A bulk or effective Young's modulus refers to a composite material formed from materials having different Young's modulus, so that the bulk or effective Young's modulus is influenced by each of the different materials and provides an overall device-level modulus.

“Flexible” refers to a material's ability to undergo a bending without fracture or permanent deformation and may be described in terms of a bending modulus.

Any of the devices may be described herein as being “mechanically matched” to skin, specifically the skin over which the device will rest. This matching of device to skin refers to a conformable interface, for example, useful for establishing conformal contact with the surface of the tissue. Devices and methods may incorporate mechanically functional substrates comprising soft materials, for example exhibiting flexibility and/or stretchability, such as polymeric and/or elastomeric materials. A mechanically matched substrate may have a modulus less than or equal to 100 MP a, less than or equal to 10 MPa, less than or equal to 1 MPa. A mechanically matched substrate may have a thickness less than or equal to 0.5 mm, and optionally for some embodiments, less than or equal to 1 cm, and optionally for some embodiments, less than or equal to 3 mm. A mechanically matched substrate may have a bending stiffness less than or equal to 1 nN m, optionally less than or equal to 0.5 nN m.

A mechanically matched device, and more particularly a substrate is characterized by one or more mechanical properties and/or physical properties that are within a specified factor of the same parameter for an epidermal layer of the skin, such as a factor of 10 or a factor of 2. For example, a substrate may have a Young's Modulus or thickness that is within a factor of 20, or optionally for some applications within a factor of 10, or optionally for some applications within a factor of 2, of a tissue, such as an epidermal layer of the skin, at the interface with a device of the present invention. A mechanically matched substrate may have a mass or modulus that is equal to or lower than that of skin.

“Encapsulate” refers to the orientation of one structure such that it is at least partially, and in some cases completely, surrounded by one or more other structures, such as a substrate, adhesive layer or encapsulating layer. “Partially encapsulated” refers to the orientation of one structure such that it is partially surrounded by one or more other structures, for example, wherein 30%, or optionally 50%, or optionally 90% of the external surface of the structure is surrounded by one or more structures. “Completely encapsulated” refers to the orientation of one structure such that it is completely surrounded by one or more other structures.

“Polymer” refers to a macromolecule composed of repeating structural units connected by covalent chemical bonds or the polymerization product of one or more monomers, often characterized by a high molecular weight. The term polymer includes homopolymers, or polymers consisting essentially of a single repeating monomer subunit. The term polymer also includes copolymers, or polymers consisting essentially of two or more monomer subunits, such as random, block, alternating, segmented, grafted, tapered and other copolymers. Useful polymers include organic polymers or inorganic polymers that may be in amorphous, semi-amorphous, crystalline or partially crystalline states. Crosslinked polymers having linked monomer chains are particularly useful for some applications. Polymers usable in the methods, devices and components disclosed include, but are not limited to, plastics, elastomers, thermoplastic elastomers, elasto-plastics, thermoplastics and acrylates. Exemplary polymers include, but are not limited to, acetal polymers, biodegradable polymers, cellulosic polymers, fluoropolymers, nylons, polyacrylonitrile polymers, polyamide-imide polymers, polyimides, polyacrylates, polybenzimidazole, polybutylene, polycarbonate, polyesters, polyetherimide, polyethylene, polyethylene copolymers and modified polyethylenes, polyketones, poly (methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin or any combinations of these.

“Elastomer” refers to a polymeric material which can be stretched or deformed and returned to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly (styrene-butadiene-styrene), polyurethanes, polychloroprene and silicones. Exemplary elastomers include, but are not limited to silicon containing polymers such as polysiloxanes including poly (dimethyl siloxane) (i.e. PDMS and h-PDMS), poly (methylsiloxane), partially alkylated poly (methyl siloxane), poly (alkyl methyl siloxane) and poly (phenyl methyl siloxane), silicon modified elastomers, thermoplastic elastomers, styrenic materials, olefinic materials, polyolefin, polyurethane thermoplastic elastomers, polyamides, synthetic rubbers, polyisobutylene, poly (styrene-butadiene-styrene), polyurethanes, polychloroprene and silicones. In an embodiment, a polymer is an elastomer.

“Conformable” refers to a device, material or substrate which has a bending stiffness that is sufficiently low to allow the device, material or substrate to adopt any desired contour profile, for example a contour profile allowing for conformal contact with a surface having a pattern of relief features. In certain embodiments, a desired contour profile is that of skin.

“Conformal contact” refers to contact established between a device and a receiving surface, specifically skin. In one aspect, conformal contact involves a macroscopic adaptation of one or more surfaces (e.g., contact surfaces) of a device to the overall shape of a surface. In another aspect, conformal contact involves a microscopic adaptation of one or more surfaces (e.g., contact surfaces) of a device to a surface resulting in an intimate contact substantially free of voids. In an embodiment, conformal contact involves adaptation of a contact surface(s) of the device to a receiving surface(s) such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface. Devices of certain aspects are capable of establishing conformal contact with internal and external tissue. Devices of certain aspects are capable of establishing conformal contact with tissue surfaces characterized by a range of surface morphologies including planar, curved, contoured, macro-featured and micro-featured surfaces and any combination of these. Devices of certain aspects are capable of establishing conformal contact with tissue surfaces corresponding to tissue undergoing movement.

“Young's modulus” is a mechanical property of a material, device or layer which refers to the ratio of stress to strain for a given substance. “Low modulus” refers to materials having a Young's modulus less than or equal to 10 MPa, less than or equal to 5 MPa or less than or equal to 1 MPa.

“Bending stiffness” is a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

“Thermal actuation state” refers to the thermal actuator that is on an off-state or an on-state. In this context, “substantially independent” refers to a position of the reference sensor that is sufficiently separated from the actuator that the reference sensor output is independent of whether the thermal actuator is on or off. Of course, the systems and methods presented herein are compatible with relatively minor effects of the actuator on the reference sensor, such as within 5%, within 1% or within 0.1% of a reference temperature when the actuator is in the on state compared to when the actuator is in the off state. Depending on specific device and tissue characteristics, this distance may be between about 10 mm and 20 mm, such as about 15 mm.

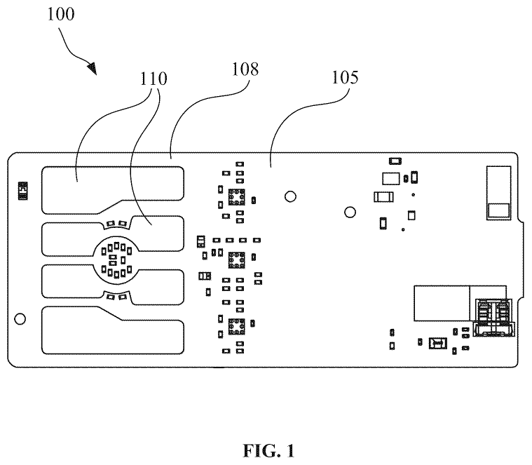

Reference is now made to FIG. 1, which illustrates an exemplary structure of a wireless thermal sensor device, such as a thermal flow sensor 100. In some embodiments, the thermal flow sensor 100 may comprise a plurality of spatial device regions, wherein different device regions have different mechanical structures imparting different mechanical properties. A device with regions of differing mechanical properties may be desirable to control mechanical strain imparted on different electronics components. For example, some electronic components may be more mechanically rigid due to their size and/or packaging, or more prone to problems due to mechanical stress than others, such as an integrated circuit (IC) component (e.g., a microprocessor, microcontroller, or other packaged IC) especially if it contains a large number of contact pads, such as greater than ten pads, and/or relatively large package area, such as greater than four square millimeters. These regions may benefit from increased overall stiffness in the device, such that strain imparted on the electronic components is reduced.

In some embodiments, the device is designed such that components that do not directly sense properties of, or provide actuation to, a biological surface may be placed in device regions of higher device stiffness, because these components do not require conformal contact to the biological surface for sensing or actuation. Other device regions may comprise elements which are smaller, more flexible, and/or more tolerant of mechanical stress. In some embodiments, these regions may also comprise sensors which are configured to sense properties during attachment to another surface. For example, another surface may be skin or other biological tissue, and sensors may be thermal sensors and/or actuators intended to measure thermal properties and/or fluid flow. In some embodiments, sensors for sensing thermal properties of or near biological tissue may obtain more sensitive, faster, and accurate readings when they are able to mechanically conform to the surface of the skin or tissue, because intimate conformation to the surface reduces thermal resistance with the underlying tissue, such as could be induced by any size of air gap.

Additionally, more mechanically compliant device regions may reduce or eliminate any change in local tissue properties induced by attaching the device to the tissue. Other sensors which benefit from intimate mechanical contact with a surface also benefit from such a construction, such as optical sensors, electrophysiology sensors such as ECG, EEG, EMG and the like, strain sensors, and other similar sensors. In other examples, surfaces may be a surface of a different device that is configured for wearing on skin, such as any manner of clothing or other wearable product, or a surface of a device configured for implantation in the body, such as any piece of a ventriculoperitoneal (VP) shunt. As such, a device with a plurality of spatial regions, wherein the spatial regions have differing mechanical properties allows for optimizing mechanics for conformal contact to a surface in one region, while retaining mechanical strength in other regions where conformal contact may be less critical to device function.

In some embodiments, a device spatial region may be a region with relatively high or relatively low flexibility. For example, a spatial region may comprise specific components which are relatively rigid, such as one or more batteries and/or packaged electronic components of more than a few square millimeters (mm²), such as greater than 4 mm², or relatively thick components, such as greater than 2 millimeters (mm) thick. In some examples, a spatial region may be made rigid by the presence of a layer intended to impart additional rigidity, such as a thicker region of a base material. For example, a stiffener layer of more than 100 micrometers (μm) of a polyimide or FR-4 layer may impart stiffness to a specified device region. A device region may be made rigid to improve robustness of specific device components, among other reasons. For example, surface mount technology (SMT) electronic integrated circuits often comprise more than four individual electronic connection points, and more than twenty contact points, wherein a stiffener may reduce the mechanical stress spread across the contact points. As another example, a different device spatial region may comprise components which are relatively small and/or with fewer electrical contacts, and less prone to reliability problems impacted by mechanical flexion. Additionally, some components, such as a sensing component, may benefit from intimate contact to the skin surface to provide optimal measurements.

In some embodiments, thermal flow sensor 100 may comprise one or more support layers to support electronics components. In some embodiments, support layers may be electrically insulating. Some examples of suitable materials for supporting electronics components include polyimide, Kapton®, composite resins, FR-4, polymer films, glass and others, with polymer films, such as polyimide or commercially available Kapton®, silicones, or polyurethanes, being preferable in regions where mechanical flexibility is required. In some embodiments, support layers may have direct bonding to patterned electrically conductive layers which form portions of the electronic circuit. For example, patterned copper on Kapton® film is a suitable construction for some flexible electronics. The patterned conductive layers may have various additional material compositions or layering, such as gold or tin plating, to provide electronic benefits such as robustness, oxidation resistance, and solderability. The layers may further support discrete electronic components which are electrically connected to the conductive layers via metallic bonding, such as by soldering or wire bonding, among other techniques.

In some embodiments, it may be advantageous to impart different mechanical design of the electronics and supporting layers in different spatial device regions. These varied structural regions can be used to impart device regions with different overall mechanical properties as described above. For example, the support layers configured to support the electronic components may be spatially patterned, as exemplified in FIG. 1. In an example where the support layer is Kapton®, the Kapton® may be a solid, continuous film in one region 105, while in a different device region 108, the Kapton® may be patterned. The patterns may comprise regions 110 of no Kapton® (e.g., patterned holes, cutouts, serpentines, or other patterns, such as greater than 5 μm thick in one region and zero thickness in other regions), or regions of varying thickness of Kapton® (e.g., Kapton® having reduced thickness in one location relative to another, such as 50 μm thick in one region and 12.5 μm thick in another). Such spatial structuring of the Kapton® provides increased device flexibility in the regions with cutouts or reduced thicknesses and may also improve the ability of the device regions to conform to three dimensional surfaces. For example, in some embodiments, it may be advantageous for a sensing region with a patterned support layer 108 to have at least 20% of the region containing no support material (such as via cutouts in Kapton®) and preferably at least 50% of the region containing no support material. Additionally, it may be advantageous for support material to be removed from regions local to individual sensors to improve mechanical conformation to surfaces, such as by having cutouts 110 in support material on at least two sides of a sensor.

In some embodiments, such as those comprising thermal sensors and/or actuators, it may be advantageous to pattern electronic supporting layers, or the electronic layers themselves, in order to reduce thermal coupling between components within the sensor. For example, polymer films such as polyimide may have a relatively high in-plane thermal conductivity, such as 2 W/(m*K) due to polymer alignment during film formation. In thermal flow sensor 100, it may be advantageous to reduce thermal coupling between a thermal actuator and some thermal sensors through the films of the device, so that thermal transport within the sensor is reduced, and is primarily through the tissue that the sensor is in contact with. This thermal transport within the sensor can be altered by removing material, such as Kapton®, between sensors and/or actuators by using cutouts 110 or patterns in the material. For example, it may be advantageous to remove electronics supporting layers between the shortest path between thermal sensors such that heat cannot conduct directly between sensors via electronic supporting layers, or electronic layers themselves.

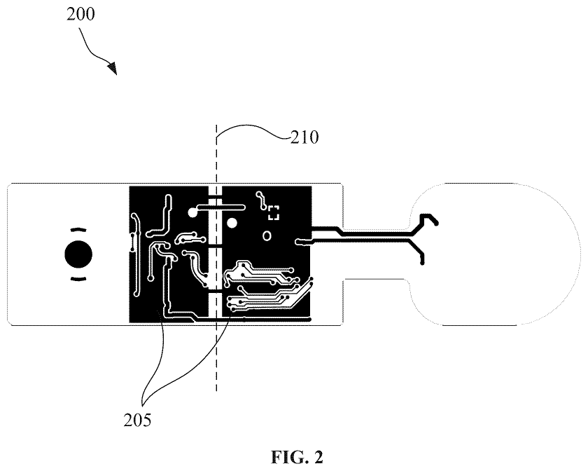

While patterning Kapton® is one example, similar structures may be applied to any other electronic support layers. Referring now to FIG. 2, electrically conductive layers may also be patterned to impart regions of differing mechanical properties. Typically, electrical grounding layers are made as solid and continuous as possible. However, these layers may be patterned to facilitate lines of increased flexibility, such as by removing >75% of the ground layer along an axis (e.g., flex axis 210) such that the stiffness along flex axis 210 is reduced, with minimal reduction in electrical efficacy of the ground plane 205.

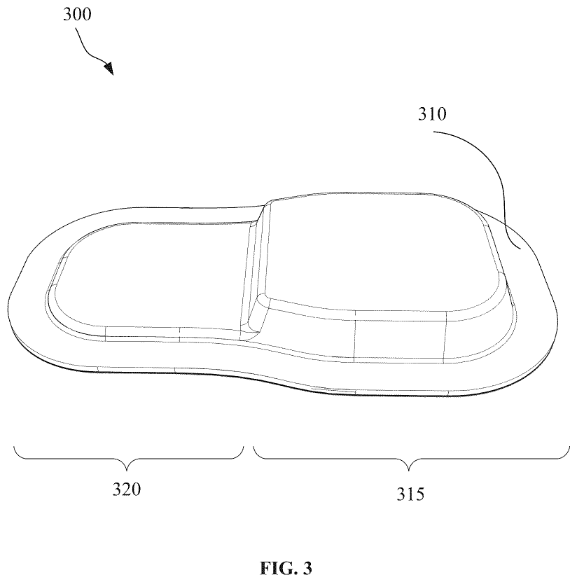

Reference is now made to FIG. 3, which illustrates an exemplary thermal flow sensor 300. Thermal flow sensor 300 may comprise an enclosure 310. In some embodiments, enclosure 310 may be used to encapsulate electronic components to prevent them from damage, to provide view comfortable device for a user, or to impart visual appeal. In some embodiments, enclosure 310 may be formed from a molded elastomeric material, such as a thermoplastic elastomer, a silicone, or a similar structure. A molded elastomeric material may be advantageous because it can be manufactured at low cost, can be highly flexible, stretchable, bonded to another portion of a sensor during assembly, or easily allows for the formation of air volumes in the final assembled device. In some examples, bonding of enclosure 310 may be enhanced by the use of adhesion promoters. In other examples, an elastomer may be directly over-molded onto another portion of the device, such as over-molding silicone over the device during assembly.

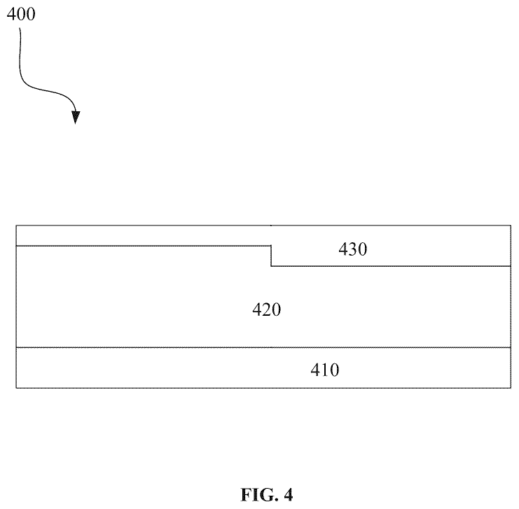

In some embodiments, the geometry of enclosure 310 may differ between different spatial device regions. For example, enclosure 310 may be taller and impart greater total device thickness in a region 315, and shorter with lower total device thickness in a region 320. For example, a region 320 may have a total thickness of less than 10 mm, and preferably less than 3 mm while a region 315 may have a total thickness greater than the thickness of region 320 and preferably greater than twice the thickness of region 320. For example, a taller region 315 may contain larger components such as a battery, larger packages electronic components such as those with areas greater than 2 mm², and/or ground planes, whereas a shorter region 320 may contain direct sensing components, such as thermal sensors or actuators, which benefit from improved contact with a surface. The reduced total device thickness will generally result in a reduced total device bending stiffness in the region with reduced overall thickness, because bending stiffness increases proportionally to third power of thickness. A reduced bending stiffness in a region with direct sensing components may be beneficial be enabling the use of lower strength, gentler adhesives to bond the sensor to a surface, such as a skin surface, while still achieving enough adhesive strength to conform the region to the surface. For example, a region 315 with an elastomeric housing, containing flexible electronic components, with a total region 315 thickness of less than 5 mm may achieve conformal contact to a skin surface with a radius of curvature of less than 10 cm, and preferably of less than 4 cm such as may occur on the skin near a human clavicle bone, by using a skin-contacting adhesive comprising a soft, casy release silicone gel adhesive of less than 3 N/25 mm adhesion strength as measured by peel adhesion. Such an embodiment may enable high sensing signal fidelity due to conformal contact to a curved surface, while also remaining comfortable to remove from a wearer. In some embodiments, a maximum ratio of bending stiffness of a sensing region to adhesion strength measured by peel adhesion may generally provide suitable adhesion to achieve conformal contact to typical anatomical curvatures, such as a ratio of less than 10 mm, and preferably less than 1 mm. In some embodiments, the height of an enclosure may be the same, but the thickness of the enclosure material may differ or be patterned in different regions. For example, as illustrated in FIG. 4, which illustrates an exemplary flow sensor device 400, one region of device 400 may have a total thickness of 8 mm, wherein a first layer 410 may be 2 mm of thickness and may comprise polymer films, adhesives, and electronics, a second layer may be 5 mm thick and may be air, and a third layer 430 may be 1 mm thick and may comprise an elastomeric enclosure (e.g., enclosure 310 of FIG. 3). In some embodiments, whereas a different region may have a thickness of 2 mm comprised of polymer films, adhesives, and electronics, 4 mm may be air, and 2 mm may be the elastomeric enclosure 310, such that the region with the thicker enclosure has a larger Young's modulus. It is to be appreciated that other thickness combinations and total thickness of device 400 may be possible as well and that the above examples are non-limiting.

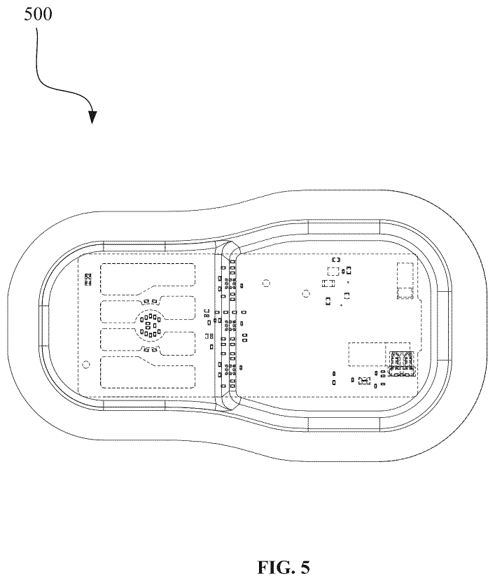

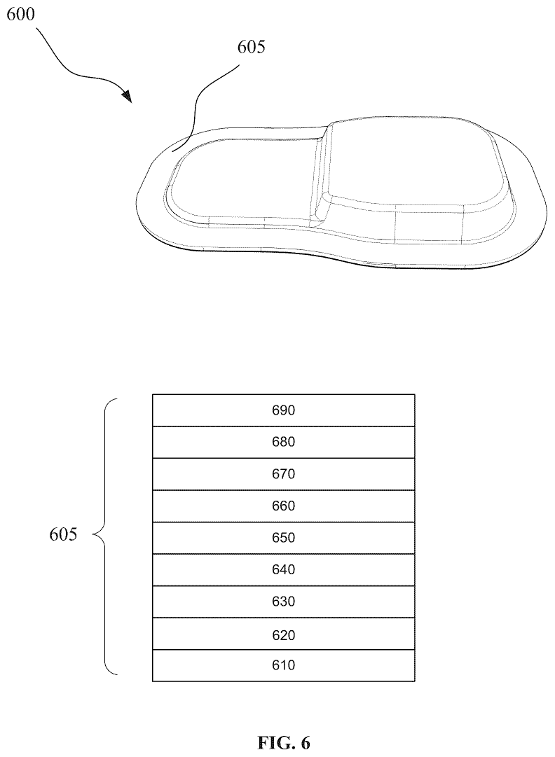

Referring now to FIG. 5 and FIG. 6, in some embodiments the enclosure may be configured such that, when bonded to one or more supporting polymer layers, the combination of the enclosure and polymer layers completely encapsulate the electronic components of the device. In other examples, such as constructions wherein the electronics are over-molded, the enclosure material may completely encapsulate the electronic components. Complete encapsulation of the electronics may provide benefits such as robust protection from ingress of particulates or fluids, which may serve as a safety benefit to reduce the possibility of electrical malfunctions. Additionally, if the enclosure and external polymer layers are soft and without sharp edges that could cut skin, such as if the enclosure is a soft elastomer such as a thermoplastic elastomer (preferably with a Durometer Shore less than 60) and the skin-contacting adhesive is silicone or silicone gel, the complete encapsulation of the electronics may also reduce chance of damaging the skin via contact with sharp edges. Additionally, complete encapsulation with materials such as thermoplastic elastomers, silicone, or other skin-safe materials imparts biocompatibility to the device, minimizing the chance of skin irritation. In a preferred example with a wireless thermal flow sensor, the thermal flow sensor may comprise a skin-contacting layer of a soft silicone gel adhesive, and an injection molded enclosure comprising a thermoplastic elastomer. A soft silicone gel adhesive of less than 1 mm thickness, and preferably less than 0.25 mm thickness such as may be achieved by a coat weight of less than 200 grams per square meter, may permit robust thermal transfer of actuation and sensing signals to the skin and underlying fluid conduits, while also imparting a soft, skin-safe surface of biocompatible material. In combination with the enclosure, which is also biocompatible, the entire surface of the device is free of sharp edges, is skin-safe, and biocompatible. An additional benefit of soft silicone adhesives such as described here is relatively high flow and wettability of the adhesive to skin. This high degree of adhesive mechanical compliance and wettability encourages an intimate thermal contact between the sensor and skin surface, free of air gaps, even under motion.

FIG. 6 illustrates a cross-section view of an outer edge 605 of an exemplary thermal flow sensor 600. In some embodiments, outer edge 605 of thermal flow sensor 600 may comprise a release liner 610, a first adhesive layer 620, a polyurethane film 630, a second adhesive layer 640, a barrier film 650, a third adhesive layer 660, a support layer 670, a fourth adhesive layer 680, and enclosure 690. It is to be appreciated that layers may be added, removed, modified, or ordered in other combinations, as appropriate. For example, a third adhesive layer 660 may be the same layer as a second adhesive layer 640, and a barrier film 650 may be the same as a polyurethane film 630 to reduce the total layers and device thickness. In some embodiments, polyurethane film 630 may be any adhesive carrier film, any adhesive layer may any suitable adhesive material, and support layer 670 may be any suitable supporting layer.

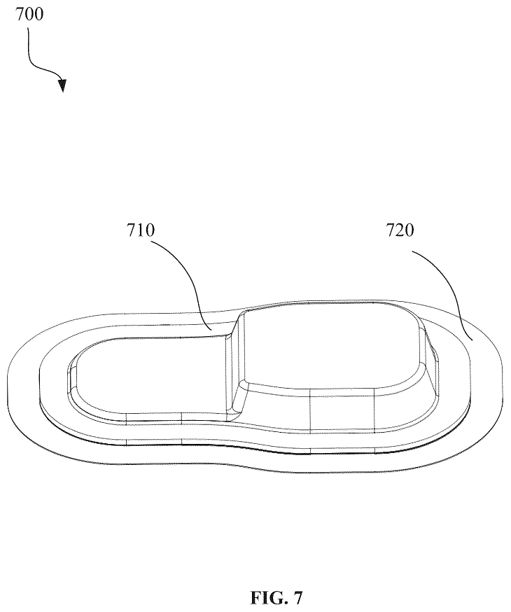

Referring now to FIG. 7, in some embodiments, such as embodiments configured for attachment of the device to living tissue or skin, the device may comprise a skin-contacting adhesive layer or laminate 720 which extends beyond the footprint of the device electronics and enclosure 710, for example 5 mm or 10 mm beyond the edge of the enclosure. This construction results in adhesive skirt or laminate 720 around the edge of the device, which may provide adhesion benefits when attached to tissue or skin. For example, when attached to skin, mechanical force on the device may induce stress at the skin-adhesive interface. It may be desirable that the skin-adhesive interface be strong under shear forces, but relatively weak under peel forces to facilitate easy removal from skin. A design employing an adhesive skirt or laminate 720 may result in a device wherein force applied to the device transfers primarily to shear stress in the adhesive, rather than peel force, because the edge of the device enclosure 710 does not reach the end of the adhesive. Such a design may be easily removed by intentionally peeling the edge of the adhesive skirt or laminate 720, which may induce peel propagation through the rest of the adhesive.

In some embodiments, a skin-contacting adhesive laminate may have a skin-contacting face and a non-skin contacting face, wherein the skin-contacting face may comprise a silicone or acrylic adhesive and a non-skin contacting face may comprise different materials in the skirt region and the device contacting region. For example, a skirt region my comprise a nonwoven media for comfort, while a device contact region may comprise an acrylic adhesive for robust bonding to a device.

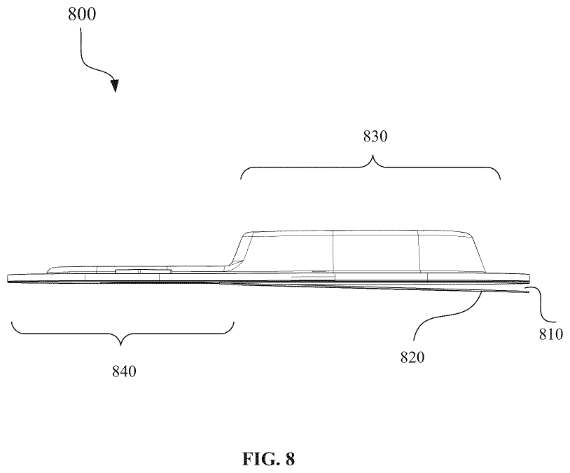

Referring now to FIG. 8, in some embodiments a skin contacting adhesive, or multilayer laminate comprising a skin contact adhesive is directly bonded to a device in one or more regions 840, and not directly bonded to the device in one or more other regions 830. For example, a skin contacting adhesive or multilayer laminate comprising a skin contact adhesive can take the form of a sheet, or laminate sheet, with a surface area, a skin contacting face, and an opposite face. The opposite face may be bonded to a surface of a device, such as by an adhesive. In areas where the skin contacting adhesive is bonded to the device (e.g., region 840), the device becomes bonded to skin when the skin contacting adhesive is adhered to skin. In other regions of the device, the opposite face of the skin contacting adhesive or adhesive laminate may not be bonded to the device. For example, the opposite face of the skin contacting adhesive or adhesive laminate may not have an adhesive layer, or may have an additional layer non-adhesive layer, such that the opposite face does not bond to the device.

In some embodiments, wherein one or more regions of a skin contacting adhesive or adhesive laminate are directly bonded to a device, and one or more other regions of a skin contacting adhesive or adhesive laminate are not directly bonded to a device, the regions without a direct bond to the device result in an adhesive flap 820 and an intermediary air gap 810. In such embodiments, when the device is applied to skin, with adhesion to skin via the skin contacting adhesive, the device will be mechanically coupled to skin in regions where the device is directly bonded to the skin contacting adhesive, and mechanically decoupled from skin in regions where the device is not directly bonded to skin. Such a construction may provide advantages in performance of the device by only constraining the device to the contours of the skin in desired regions. For example, the adhesive may be directly bonded to the device in device regions of differing mechanical properties, such as the adhesive being directly bonded to the device in device regions of relatively high flexibility, and not directly bonded to the device in device regions of relatively low flexibility (or high rigidity).

In some embodiments, sensing and actuating components may be located in a region of high device flexibility, and also direct bonding between the skin contacting adhesive and the device. The non-sensing components, including a microprocessor, a battery, or other electronic components may be located in a region of low device flexibility without direct bonding to skin contacting adhesive. In this configuration, the sensing and actuating components are able to make intimate contact to skin, with low adhesive force, because the low flexibility region of the device do not need to conform to the surface of the skin. Such a construction enables an overall device construction that accomplishes both intimate conformal skin contact in critical regions for sensing and actuating, and also a construction allowing for use of rigid, inflexible components.

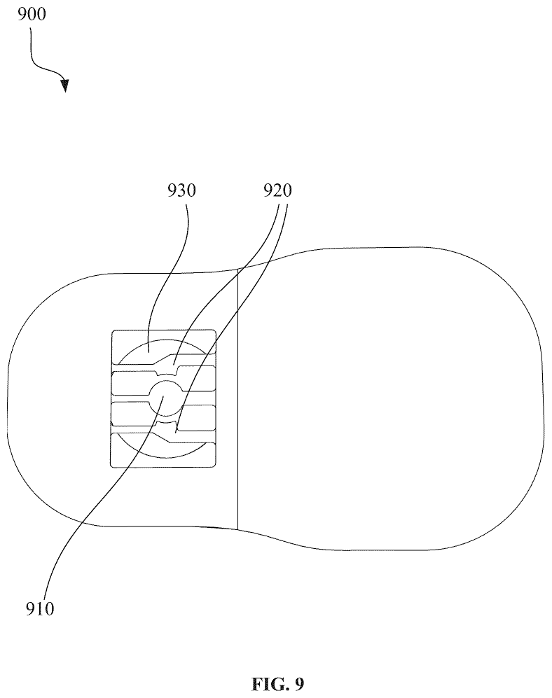

Referring now to FIG. 9, in some embodiments an insulating media 930 may be disposed on and/or around sensors 920 and/or actuators 910, such as thermal sensors and/or actuators, or such as thermal flow sensors and/or actuators. In some embodiments, insulating media 930 may improve the measurement signal quality of the thermal sensor by reducing the effect of environmental effects, such as air flow, on sensor signals. For example, a skin-mountable flow sensor may be configured with insulated media 930 such that when the sensor is applied to skin, the sensors are disposed between the skin and insulating media, thereby enhancing the effect of signals propagating from the skin.

In the context of this disclosure, an insulating media refers to a media which has a lower thermal conductivity than that of a skin, and preferably lower than 0.2 W/(m*K), and more preferably lower than 0.05 W/(m*K). For example, an insulating media may be an enclosure air space, or an air composite such as a foam insulation. In some embodiments, a foam insulation may comprise an adhesive surface between the foam and the thermal sensors and/or actuators, such that the foam can be cut from a sheet of foam with an adhesive face and applied to the sensors and/or actuators at low cost during manufacture.

In some embodiments, an enclosed air space may be formed by the boding of an enclosure over the sensors and/or actuators, such that air currents external to the device enclosure do not create substantial thermal convective heat transfer to or from the sensors and/or actuators. An insulating media may have several advantages including, but not limited to, for thermal sensors and actuators by minimizing thermal transport effects from the external environment, thereby reducing thermal noise, for other sensors, actuators, and other electronic components by providing mechanical protection, reduction in electrical noise (preferably by use of an electrically conductive insulating media), or separation from other components.

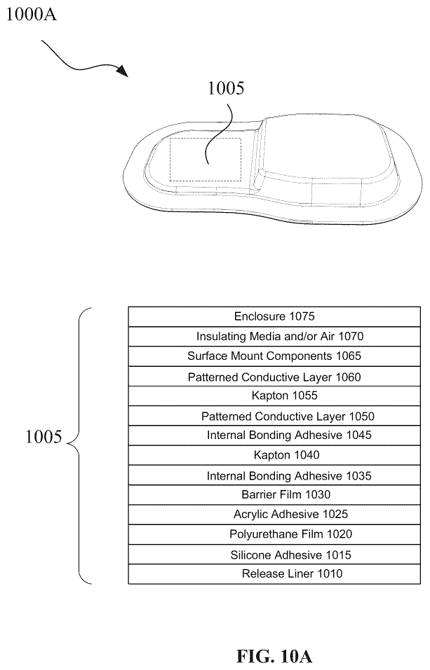

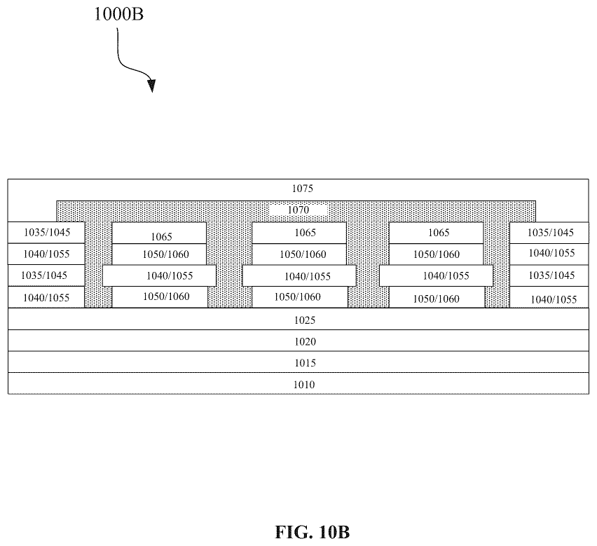

Referring now to FIGS. 10A and 10B, in some embodiments a device may have a layered construction comprising multiple materials, including adhesives, electrically nonconductive layers, electrically conductive layers, and elastomers. Such layering structures may be useful to enable a sensor which may be protected from environmental effects, insulated, able to effectively comprise electronic components, and patterned to enable flexibility in desired regions. In some embodiments, the sensor may include mechanical supporting layers for handling during manufacturing and electrical insulation, adhesive bonding of the device components, and adhesive bonding to skin.

FIG. 10A illustrates a cross-section view of a region 1005 of an exemplary thermal flow sensor 1000A including functional electronic layers. In some embodiments, thermal flow sensor 1000A may comprise a release liner 1010, a first adhesive layer 1015, a polyurethane film 1020, a second adhesive layer 1025, a barrier film 1030, a third adhesive layer 1035, a first Kapton® support layer 1040, a fourth adhesive layer 1045, a first patterned conductive layer 1050, a second Kapton® support layer 1055, a second patterned conductive layer 1060, surface mount electronic components 1065, insulating media and/or air 1070, and enclosure 1075. It is to be appreciated that layers may be added, removed, modified, or ordered in other combinations, as appropriate. For example, a third adhesive layer 1035 may be the same layer as a fourth adhesive layer 1045, and a barrier film 1030 may be the same as a polyurethane film 1020 to reduce the total number of layers and device thickness. In some embodiments, polyurethane film 1020 may be an adhesive carrier film, an adhesive layer, or a suitable adhesive material, and support layers 1040 and 1055 may be a suitable supporting layer. In some embodiments, only one of patterned conductive layers 1050 and 1060 may be present.

FIG. 10B illustrates a cross-section view of a region 1005 of an exemplary thermal flow sensor 1000B including functional electronic layers and cutouts or patterns. The cutouts or patterns illustrated in FIG. 10B may be useful for increasing mechanical flexibility or improving thermal isolation between components. In some embodiments, thermal flow sensor 1000B may comprise a release liner 1010, a first adhesive layer 1015, a polyurethane film 1020, a second adhesive layer 1025, a first Kapton® support layer 1040, a fourth adhesive layer 1045, a first patterned conductive layer 1050, a second Kapton® support layer 1055, a second patterned conductive layer 1060, surface mount electronic components 1065, insulating media and/or air 1070, and enclosure 1075. It is to be appreciated that layers may be added, removed, modified, or ordered in other combinations, as appropriate. In some embodiments, polyurethane film 1020 may be any adhesive carrier film, any adhesive layer may any suitable adhesive material, and support layers 1040 and 1055 may be any suitable supporting layer. In some embodiments only one of patterned conductive layers 1050 and 1060 may be present.

In some embodiments a reduced bending stiffness in a region 1005 with direct sensing components may be beneficial for enabling the use of lower strength, gentler adhesives to bond the sensor to a surface, such as a skin surface, while still achieving enough adhesive strength to conform the region to the surface. For example, a region 1005 wherein: layers 1015, 1020 and 1025 combined are less than 0.5 mm thick; layers 1035, 1040 and 1045 combined are less than 0.1 mm thick; layers 1050, 1055 and 1060 combined are less than 0.2 mm thick; and layers 1065, 1070 and 1075 combined are less than 3 mm thick enables a direct sensing region 1005 of less than 10 mm total thickness, and preferably less than 4 mm total thickness, such that the bending stiffness in the region, when combined with an enclosure material with a low Young's modulus, such as an elastomer with a Young's modulus of less than 5 MPa, and spatially patterning of layers 1050/1060, 1040/1055 and 1065, is suitable for adhesion to a region of human skin with a radius of curvature of less than 10 cm, and preferably of less than 4 cm such as may occur on the skin near a human clavicle bone, by using a skin-contacting adhesive 1010 comprising a soft, easy release silicone gel adhesive of less than 3 N/25 mm adhesion strength as measured by peel adhesion. Such an embodiment may enable high sensing signal fidelity due to conformal contact to a curved surface, while also remaining comfortable to remove from a wearer. In some embodiments, a maximum ratio of bending stiffness of a sensing region to adhesion strength measured by peel adhesion may generally provide suitable adhesion to achieve conformal contact to typical anatomical curvatures, such as a ratio of less than 10 mm, and preferably less than 1 mm.

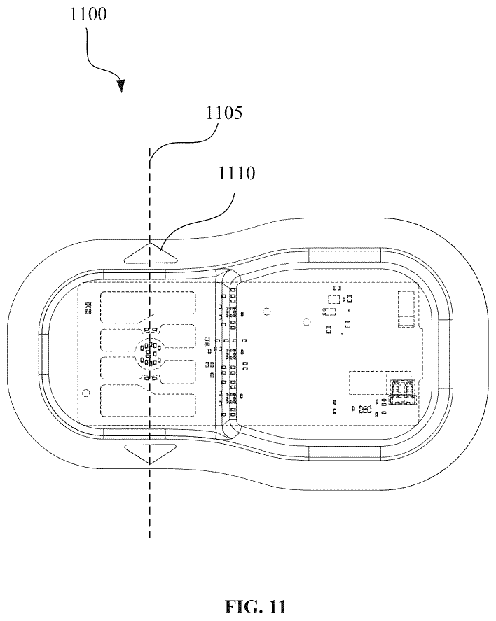

Referring now to FIG. 11, which illustrates a top view of an exemplary thermal flow sensor 1100, consistent with disclosed embodiments. In some embodiments, thermal flow sensor 1100 may comprise a patterned surface 1110, visible by an external view of a device, indicating one or more lines 1105 which correspond to an alignment axis of one or more sensors and/or one or more actuators. For example, a patterned surface may comprise raised features, lowered features, embossed features, colored features, or any similar manner of readily visible features. In an example, a device may comprise a thermal actuator and one or more thermal sensors where a line drawn between the thermal actuator and one or more thermal sensors would align with a line drawn between the shaped edge features, such that the device may be easily placed to align the actuator and sensors to a measurement region of interest, such as a fluid conduit beneath a skin surface. Such features which improve the ability to align a sensor to a region of interest may be useful for improving the measurement quality and ese of use of a sensor.

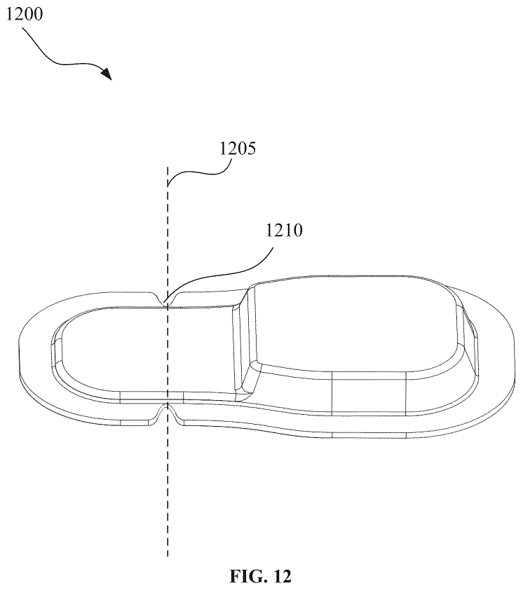

Referring now to FIG. 12, which illustrates an exemplary thermal flow sensor 1200, consistent with disclosed embodiments. In some embodiments, thermal flow sensor 1200 may comprise a shaped edge of an enclosure, wherein the shaped edge has visible features 1210 which correspond to an alignment axis 1205 of one or more sensors and/or one or more actuators. For example, a device may comprise a thermal actuator and one or more thermal sensors where a line drawn between the thermal actuator and one or more thermal sensors would align with a line drawn between the shaped edge features, such that the device may be easily placed to align the actuator and sensors of a measurement region of interest. Such features which improve the ability to align a sensor to a region of interest may be useful for improving the measurement quality and ese of use of a sensor.

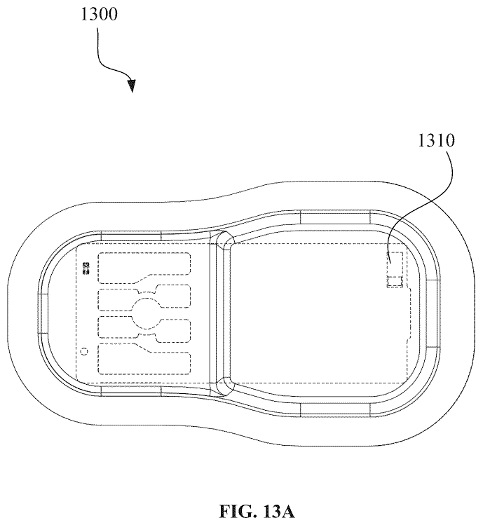

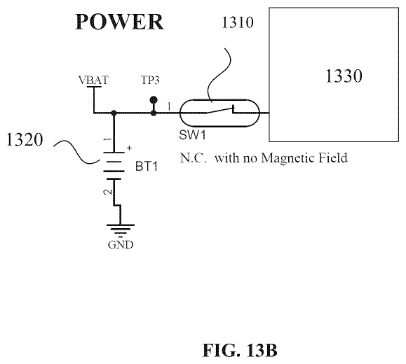

Referring now to FIGS. 13A and 13B, in some embodiments a device 1300 may comprise a mechanism for disrupting an electrical connection between a power source and at least one or more other electronics elements in the device. For example, a device may comprise a power switch 1310 configured to open or close a circuit between a battery 1320 and the other electronics 1330 in a device, wherein a battery 1320 may be an individual battery or a plurality of batteries comprising protection circuitry, or a battery pack. In some embodiments, power switch 1310 may be a magnetic switch, activated or deactivated by a magnetic field. In some embodiments, magnetic switch 1310 may be configured such that the switch is normally closed with no magnetic field present, and normally open in the presence of a magnetic field, such a magnetic field will cause the device 1300 to be powered ‘off’. Such a configuration may enable a device which can be packaged with a magnet, such that the device is powered “off” in packaging and becomes powered “on” upon removal from packaging. Magnetic switches may allow controlling the switch such as, turning on or off, without any mechanical force on the switch, thereby enabling a soft, fully enclosed device construction.

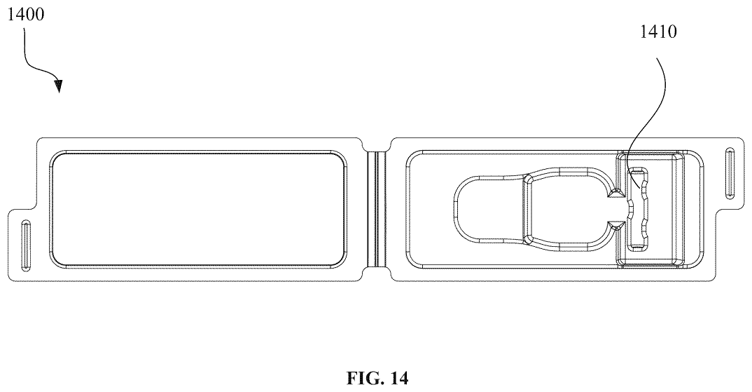

In some embodiments, a device 1400 with a magnetic switch 1410 may benefit from a packaging construction comprising a magnet, such as shown in FIG. 14, wherein the device fits into the packaging such that the magnet opens the magnetic switch, preventing power to the device. Such a construction may ensure that the device is not under power while in the packaging, and immediately becomes powered upon removal from the packaging. Such a construction may enable a significantly improved shelf-life of a sensor without requiring an end user to charge the device before use, while retaining a fully enclosed device construction.

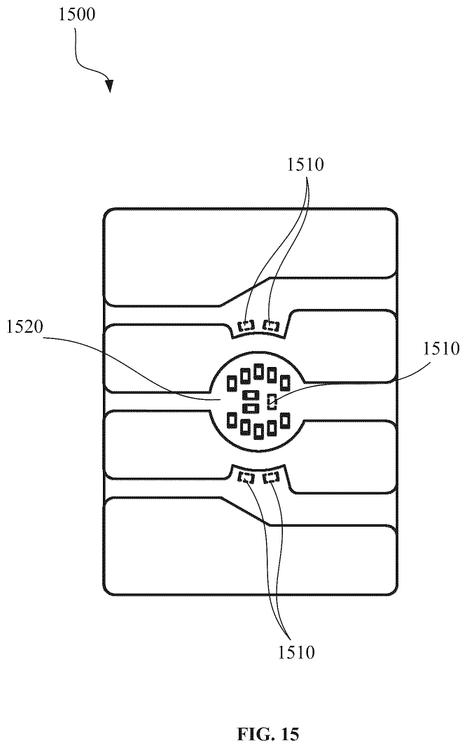

Referring now to FIG. 15, which illustrates an exemplary configuration 1500 of thermal actuators and thermal sensors. In some embodiments, a device may comprise a thermal actuator 1520 and one or more temperature sensors 1510 located such that the temperature sensors 1500 monitor the temperature of the thermal actuator 1520. For example, a temperature sensor, or a plurality of temperature sensors, may be located within an array of resistors comprising a thermal actuator. A temperature sensor may be any element which undergoes a change in an electronic property in response to a change in temperature. In a preferred example, a temperature sensor for monitoring the temperature of a thermal actuator is an element which changes electrical resistance in response to a change in temperature, such as a thermistor, such as a positive temperature coefficient thermistor or a negative temperature coefficient thermistor. For example, a reading from the temperature sensor, either directly or indirectly, may be used by a microcontroller to turn a thermal actuator on or off.

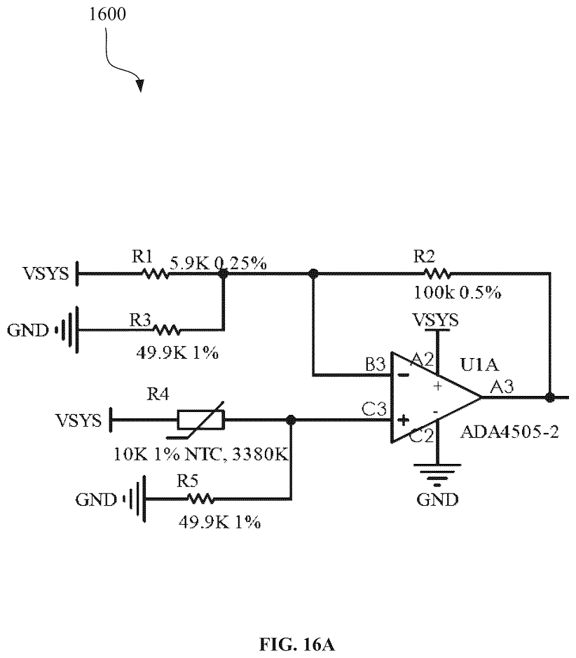

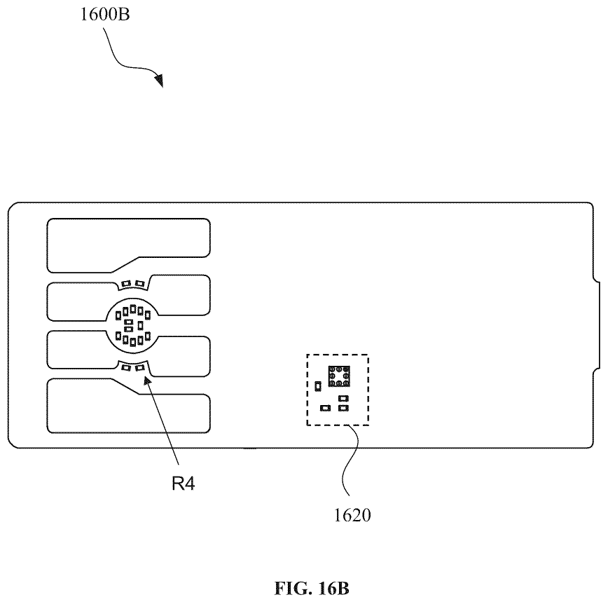

Reference is now made to FIG. 16A, which illustrates an exemplary circuit representation 1600 for a temperature sensor, consistent with embodiments of the present disclosure. In some embodiments, circuit 1600 may be configured such that when the circuit is powered, the voltage across the temperature sensor forms a portion of a voltage comparator circuit via an operational amplifier. Tuning of the operational amplifier circuit allows for tuning of the temperature range and gain of the temperature sensor reading, wherein the voltage output of the operational amplifier is wired to an input of a microprocessor, such that the voltage output of the operational amplifier corresponds to the temperature of the temperature sensor via a calibration.

As shown in FIG. 16B, an exemplary device configuration 1600B may enable placing a very small temperature sensor R4, such as in a conventional 0201 electronic package, in a different device location from the other components 1620. For example, such a construction may enable placement of a temperature sensor R4 in a device region with spatial patterning for high mechanical flexibility and thermal isolation, while placing all other components in a device region with low flexibility away from direct sensing components.

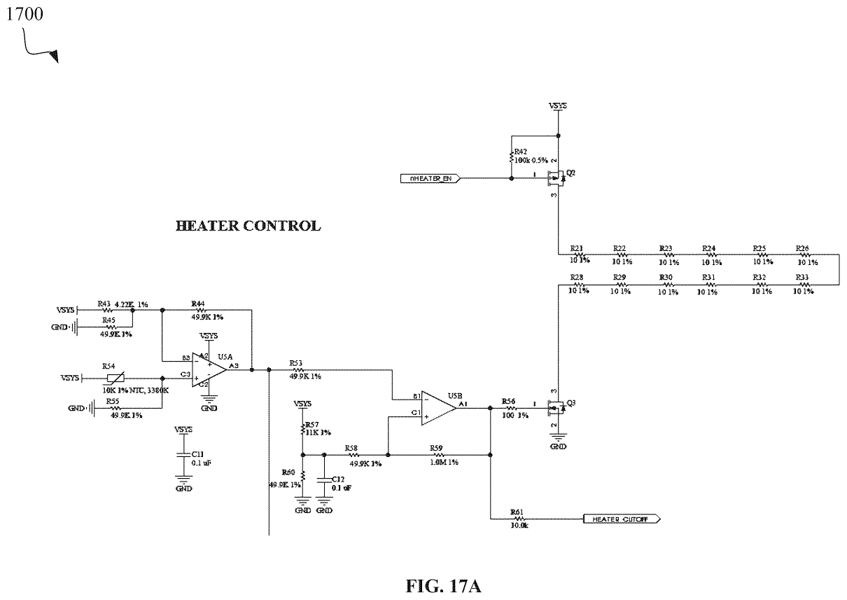

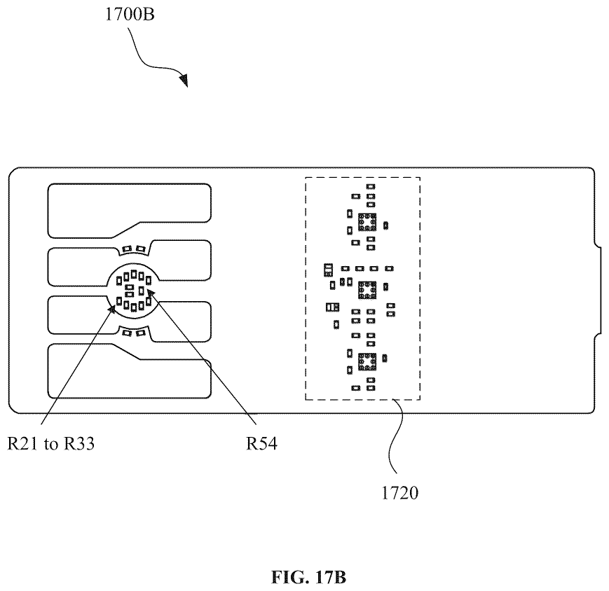

In some embodiments, a temperature sensor for monitoring a temperature of a thermal actuator may be used as part of a control system for altering power to a thermal actuator. For example, referring now to FIG. 17A, which illustrates an exemplary circuit representation for a temperature sensor for monitoring a temperature of a thermal actuator may be designated R54 and a thermal actuator may be comprised of an array of resistors designated R21 to R33. In an example, such a device configuration may enable placing very small resistors R21 to R33, such as in a conventional 0201 electronic packages, in a different device location from the other components. For example, such a construction may enable placement of resistors R21 to R33 in a device region with spatial patterning for high mechanical flexibility and thermal isolation, while placing all other components in a device region with low flexibility away from direct sensing components. R21 to R33 may produce heat when electrical current passes through them, wherein the electrical current is produced when a voltage differential is imparted across the array, wherein the voltage differential is the voltage difference between VSYS and GND.

Additional element may be wired in series with the thermal actuator array (R21 to R33) and the voltage or ground source. For example, a transistor, such as designated Q3, may be wired in series with the thermal actuator to effectively serve as a control switch. In an ‘on’ state, Q3 has a resistance significantly lower than the thermal actuator and allows significant current flow through the thermal actuator (for example, enough current to generate heat, preferably with a power greater than 0.5 milliwatts per square millimeter (mW/mm²) and less than 10 mW/mm²). In an ‘off’ state, Q3 has an electrical resistance which is substantially higher than the thermal actuator, preferable at least ten times higher, more preferably at least one thousand times higher, such that the current flowing through the thermal actuator is too low to generate substantial heating. The state of Q3 may be controlled by the application of a gate voltage to Q3, wherein the gate voltage controls whether Q3 is in the ‘on’ or ‘off’ state. In some embodiments, the gate voltage to an element such as Q3 may be the output voltage of a comparator circuit, wherein the comparator circuit comprises a plurality of electrical elements including the temperature sensor of the thermal actuator, other resistors, and one or more comparators such as an operational amplifier.

In some embodiments, as illustrated in FIGS. 17A and 17B, the circuit 1700 can be arranged such that the output voltage of a comparator circuit varies depending on the temperature of the temperature sensor for monitoring the temperature of the thermal actuator (via a resistance change of the temperature sensor as a result of temperature changes). The voltage output of the comparator circuit may be wired to a transistor, such as Q3, such that the ‘on’/‘off’ state of Q3 is controlled by the output of the comparator circuit. In such a configuration, power to the thermal actuator is effectively prevented depending on the temperature of the thermal actuator, because the circuit may be configured such that Q3 is the ‘off’ state when the temperature sensor R54 is in a pre-determined temperature range. Such a configuration has an advantage of being an entirely analog control circuit, such that it is independent of any microprocessor execution or software execution steps. This may be advantageous to a thermal sensor, such as a thermal flow sensor, by providing a completely analog thermal control mechanism, such as for safety purposes, which will function correctly independent of any software elements. For example, the control circuit may be configured such that power to the thermal actuator is prevented when the temperature sensor R54 exceeds a value, preferably a value below which damage to tissue, such as skin, is known to occur.

In some embodiments, a temperature cutoff circuit may be configured such that Q3 is ‘off’, thereby preventing power to the thermal actuator, when the temperature of the thermal actuator exceeds 48° C. if the actuator is on for less than 10 minutes, or less than 43 degrees Celsius (° C.) if the actuator is on longer than 10 minutes. Preferably, the temperature cutoff circuit is configured to prevent power to the thermal actuator below the specified temperature even when all circuit elements in the comparator circuit are at the edge of their tolerance range. For example, a resistive element specified as 49,900 Ohms (Ω) will not have a resistance of exactly 49,900 Ω due to manufacturing tolerances. A temperature cutoff circuit can thereby be designed with a selection of electrical components whereby the temperature cutoff will occur at or below the desired temperature even if all elements in the circuit are at the edge of their specified value range. In an example, this may result in a circuit that, when all circuit elements are at their nominal specified value, power to the thermal actuator is prevented when the temperature sensor R54 reaches 46.5° C., such that even if a device is manufactured with all circuit elements at the edge of their specified tolerance (resulting in a thermal cutoff occurring at a higher temperature), the power to the thermal actuator is still cutoff below a temperature of 48° C. While it may be advantageous to select circuit elements with the smallest possible tolerance range, this can create practical challenge related to cost and part availability, whereby parts with tighter tolerances are more expensive and result in a less flexible design. However, on the other hand, if the tolerances are too wide, the nominal temperature cutoff point may have to be too low, resulting in reducing sensor signal strength, to account for the possible range of values. It is therefore advantageous to specify circuit element parameters that result in an acceptable range of possible cutoff temperatures, while still providing low cost, readily available components.

In some embodiments, as shown in FIG. 17A, circuit 1700 may be designed with resistive elements with a 1% range in their resistance value to result in an acceptable nominal temperature cutoff value of >46° C., such that even if every resistive element in the circuit has a resistance 1% away from its specified resistance, the temperature cutoff will still occur below 48° C. The tolerance of the temperature sensor (R54 in this example) is of particular importance, wherein a tolerance of 5% would require a much lower nominal temperature cutoff than a tolerance of 1%. While specific parameter values of circuit elements are described in the examples, the specific values of the resistors, temperature sensor resistance, and comparator parameters can be varied in numerous ways to result in similar effects. For example, many configurations of resistor values in a comparator circuit can be used to result in a temperature cutoff of 48° C., depending on the selection of the temperature sensor. Similarly, a control element such as transistor Q3 can be selected such that ‘on’ and ‘off’ can occur at either high or low gate voltages (for example, n-channel or p-channel MOSFETS). In some embodiments, the entire VSYS voltage rail may be configured such that it can enabled or disabled, for example by being configured as a variable output from a voltage regulator. For example, a voltage regulator may comprise a plurality of voltage outputs, wherein at least one output provides constant voltage to a microprocess, and at least one other output can be turned on our off based on a signal from a microprocessor. For example, the variable voltage output may supply voltage to a thermal actuator, to sensors such as temperature sensors, to voltage comparator circuits, to operational amplifiers, or all of them together.

In some embodiments, a device may comprise a plurality of elements capable of altering or preventing power to a thermal actuator. These elements may provide added measurement control, added safety, or both. For example, a circuit may comprise an additional transistor Q2, whereby the gate voltage and therefor the ‘on’/‘off’ state is controlled and delivered by a microprocessor, wherein the microprocessor comprises a set of instruction (for example, software) for changing the ‘on’/‘off’ state of Q2. For example, if the microprocessor also receives signals for the temperature sensor for monitoring the thermal actuator, the microprocessor may determine a temperature value of the thermal actuator by comparing the received signal to a set of calibration values or equations. The microprocessor may then turn Q2 to the ‘off’ state if the measured temperature exceeds a threshold. In another example, the microprocessor may comprise instructions to turn Q2 to the ‘off’ state for any other reason, such as a loss of a Bluetooth connection with another device, such as a handheld computing device used to control a thermal flow sensor. In such a configuration, power may be prevented to the thermal actuator in any scenario that turns either Q3 or Q2 to the ‘off’ state which, in some embodiments, may provide a single-fault protection thermal failsafe system, wherein there are two or more independent mechanisms for preventing power output to the thermal actuator. Such single-fault protections may provide improved safety to a user compared to configurations which rely on a single mechanism for preventing power to the thermal actuator. In some embodiments Q3 represents a hardware-controlled thermal failsafe mechanism, as its function is independent of any software-controlled process, and Q2 represents a software-controlled thermal failsafe mechanism because it is controlled by a microprocessor. In other embodiments, the memory comprising instructions for controlling Q2 may be a part of additional components of a system, such as a sensor control device, which is in wireless communication with the microprocessor of the thermal flow sensor. In some embodiments, as shown in FIG. 17B, an exemplary device configuration 1700B may enable placing a very small temperature sensor R4, such as in a conventional 0201 electronic package, in a different device location from the other components 1720.

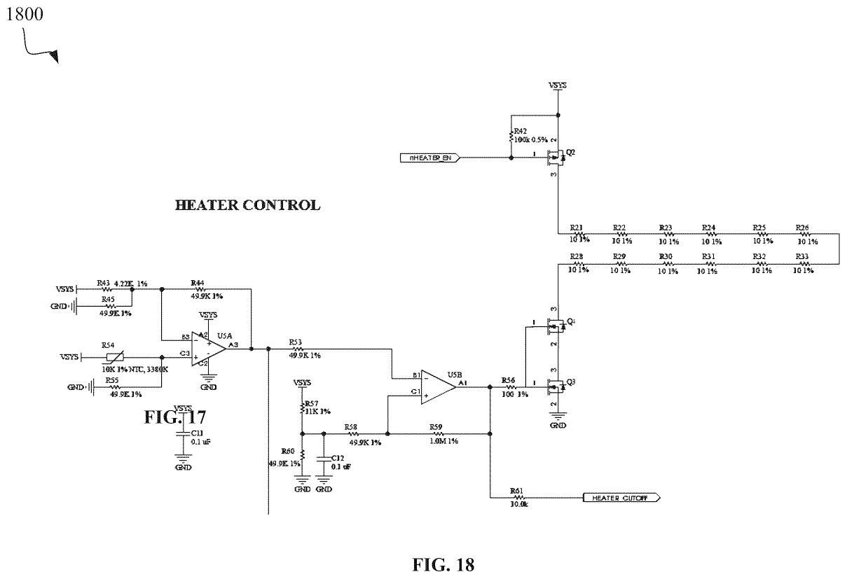

Reference is now made to FIG. 18, which illustrates an exemplary circuit 1800 including a redundant hardware thermal failsafe circuit, consistent with disclosed embodiments. In some embodiments, a device may include redundant thermal actuator control elements. For example, a device may comprise two discrete transistor elements Q3 and Q4 elements whereby the source and drain of the elements are wired in series and the gate voltage is wired in parallel, such that even if one element fails, the redundant element still prevents power to the thermal actuator.

In some embodiments, it may be advantageous for a thermal actuator to deliver an approximately constant power or approximately constant temperature, such as a power or temperature within a range. For example, a device may be configured to supply an approximately constant power to a thermal actuator via a voltage regulator. In an example, a device comprises a power source such as a battery operating at greater than 2.5 V, and preferably greater than 3.5 V, and a voltage regulator configured to receive voltage from the battery and output a lower voltage, such as 2.3 V. The output voltage is then delivered to the thermal actuator, such that 2.3 V is delivered consistently to the thermal actuator even when the battery voltage decreases. When the voltage regulator is used in combination with a thermal actuator wherein the thermal actuator comprises one or more resistors that have an approximately constant resistance within the temperature range of operation, preferably by having a temperature coefficient of resistance less than +/−1,000 ppm/° C., more preferably less than +/−250 ppm/° C., the voltage and resistance of the thermal actuator remain approximately constant through the operation of the device, resulting in a constant power delivery throughout the operation of the device. Constant power delivery may be advantageous in a thermal sensor, such as a thermal slow sensor, by providing consistent actuation power across multiple measurements and devices, resulting in consistent sensing signals. In alternative embodiments, a circuit may be configured to deliver constant current to a thermal actuator, via a constant current delivery configuration, instead of a constant voltage. In yet other embodiments, the device may be configured to maintain the thermal actuator at a constant temperature by continuously adjusting the power delivered to the thermal actuator. For example, a microprocessor may receive signals from a temperature sensor as an input, and adjust the power delivered to a thermal actuator to maintain a constant temperature of a temperature sensor. For example, such a device may be configured to maintain a thermal actuator at a constant temperature of 48° C. or 43° C. In yet other embodiments, a positive temperature coefficient (PTC) of resistance element may be configured electrically in series with the heating elements of a thermal actuator, and physically placed within or near to a thermal actuator. Such a configuration results in an increase in the resistance of the PTC element as the thermal actuator temperature increases. As the PTC resistance increases, the voltage differential across the thermal actuator decreases, such that the delivered power decreases. Such a configuration enables an entirely analog system for maintaining a maximum temperature and approximately constant temperature of a thermal actuator.

In some embodiments, it may be advantageous to provide a thermal power, via a thermal actuator, of a specific value, or within a range of values. For example, it may be advantageous to supply a power such that a temperature rise greater than 0.5° C., and preferably greater than 1.0° C., and more preferably greater than 2.0° C., occurs in temperature sensors adjacent to the thermal actuator within five minutes of beginning to supply power to the thermal actuator when the device is applied to human skin. It may further be advantageous if the same device results in the thermal actuator temperature remaining below the thermal cutoff temperature (for example, 48° C. or 43° C.) of the circuit under normal operation when the device is applied to skin. As one example, such a condition can be achieved with a circular thermal actuator of 2 mm radius, a cumulative resistance of 120 Ω (for example, by being comprised of ten individual 12 Ohm surface mount resistors of 0201 package size), and voltage delivered by a voltage regulator of 2.3 V, resulting in a total power of 44 milliwatts or 3.5 mW/mm². Such a construction, when applied to skin at ambient temperatures below 25° C., may result in a temperature of the thermal actuator after 5 minutes of constant power of below 46.5° C., and a temperature increase of adjacent temperature sensors of greater than 1.5° C. These conditions, when combined with other elements of construction described herein, enable a measurement that remains below the threshold of human sensation or damage to skin, while providing temperature signals with a relatively high signal to noise ratio. However, many other configurations can also provide suitable measurements. For example, a different arrangement of voltage delivered to the actuator and cumulative resistance of the actuator can result in the same delivered power. Alternatively, a higher or lower power may also be suitable, such as between 10 milliwatts and 100 milliwatts, so long as the parameters result in a temperature increase of the thermal actuators within safe levels for human skin, and also result in a measurable temperature increase upon thermal actuation.

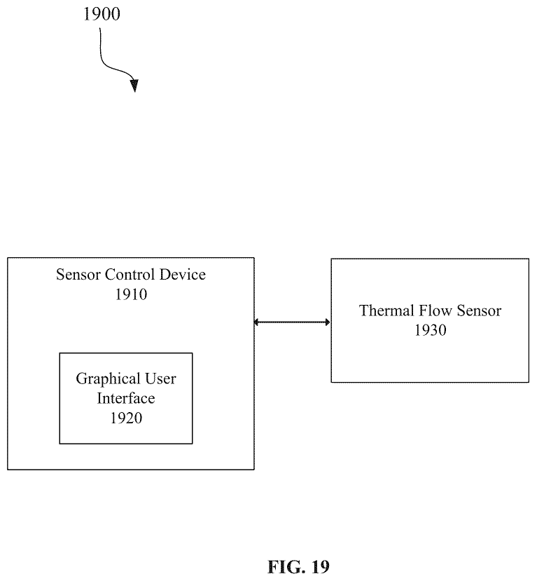

Reference is now made to FIG. 19, which illustrates an exemplary arrangement 1900 of a sensor control device comprising a user interface 1920, an application software, and wireless communication circuitry such as Bluetooth Low Energy may be configured to provide instructions to and receive data from a thermal sensor 1930, such as a thermal flow sensor for monitoring flow parameters through a ventriculoperitoneal shunt. For example, sensor control device 1910 may be a handheld computing device such as a tablet computer or a smartphone.

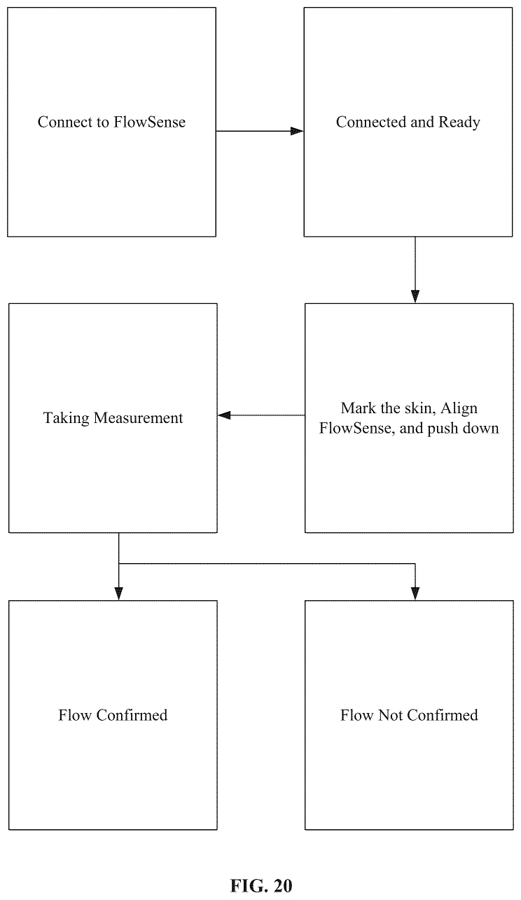

In some embodiments, sensor control device 1910 may provide instructions to a user to operate a device, via a graphical user interface 1920. The instructions may include, but are not limited to, wirelessly connect to a sensing device, apply visual marks to a user with an implanted shunt to indicate where the shunt in, apply the sensing device to the user such that the alignment marks on the sensing device align the marks on the user, press down on a portion of the sensing device to increase the chances of good adhesive contact, ensure that the user is siting up, and begin a measurement, wherein a measurement may comprise steps of: receiving sensor data from a thermal flow sensor, executing a set of instructions based at least in part on the received sensor data to determine that a measurement can proceed to a next step, send a command to supply power to a thermal actuator (for example, by changing the voltage applied to a transistor gate voltage that controls the flow of current to a thermal actuator), execute a set of instructions based on received sensor data after the thermal actuator begins receiving power to determine if a measurement will proceed, receive additional sensor data, and after some amount of time, send a command to disable the supply of power to the thermal actuator. In some embodiments, the sensor control device may comprise additional instructions to determine a parameter related to a flow of fluid and display the result to a user. For example, the parameter related to a flow of fluid may be whether flow is “Confirmed” or “Not Confirmed” through an underlying implanted shunt, or a flow rate of a fluid in an underlying implanted shunt, as illustrated in FIG. 20.

In some embodiments, sensor control device 1910 may receive data from thermal flow sensor 1930 indicative of the total amount of the time that the thermal actuator has been turned on, and further prevent further use of the device if the amount of time that the heater has been turned on exceeds a predetermined threshold, for example 25 minutes. Optionally, the sensor control device 1910 may also receive from a thermal flow sensor 1930 a set of parameters defining a calibration between digitized voltage values and a temperature of each temperature sensor in thermal flow sensor 1930, wherein the sensor control device 1910 uses the parameters to convert sensor data into temperature data for further analysis. In some embodiments, either a sensor or a sensor control device 1910 may comprise a microprocessor, a memory and set of instructions which may be executed to undergo any manner of checks of sensor data to determine that a measurement may proceed. For example, if sensor data noise exceeds a threshold, a measurement may not be permitted to proceed, and an error message may be displayed to the user. Additionally, or alternatively, remedial actions may also be displayed to the user. In other examples, if a sensor signal is determined to be out of range, a measurement may not be permitted to proceed, and an error message may be displayed to a user.

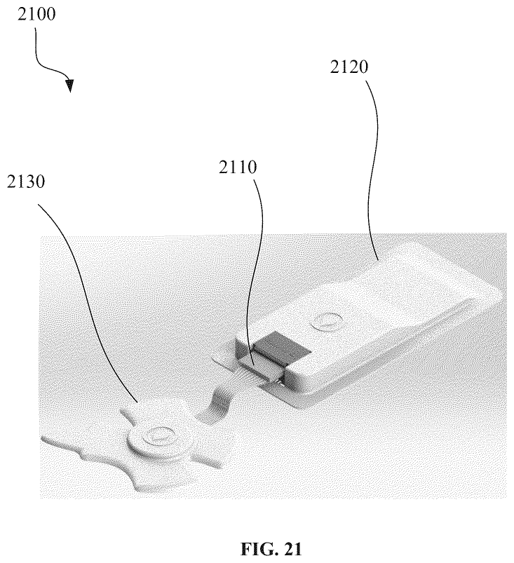

Reference is now made to FIG. 21, which illustrates an exemplary thermal flow sensor 2100, consistent with some disclosed embodiments. Thermal flow sensor 2100 may comprise a plurality of physical components for attachment to skin, including some reusable and some disposable components, wherein any of the components may utilize any combination of disclosures herein. For example, thermal flow sensor 2100 may comprise a releasable electromechanical connection 2110 between a sensing module 2130 and a processing module 2120. In some embodiments, sensor module 2130 may comprise components for direct sensing and actuating of skin, and the processing module 2120 may comprise components for providing power and controlling and receiving signals from the sensors and actuators. Such a construction may provide advantages to the end user by enabling repeated use of the expensive components of a sensor (e.g., the processing module 2120) and disposable use of the least costly components of a sensor (e.g., the sensor module 2130). For example, the sensor module 2130 may comprise only passive electronic components such as resistors, thermistors and capacitors, among other components and the processor module 2120 may comprise integrated circuits, embedded software, and a battery, among other things. It may be beneficial to dispose of the sensor module 2130 after one or several uses to avoid signal changes that may result from degradation, contamination, or fouling of the adhesive directly beneath the sensor or actuator components. Use of a new sensor module may ensure a clean adhesive surface in the most critical regions near the sensors and actuators without requiring the end user to replace the adhesive, which may be prone to error by the end user. The processor module 2120 may be less sensitive to fouling or poor replacement of the adhesive, as it does not sense or actuate through the adhesive, and may therefore be more suitable to a reusable module.

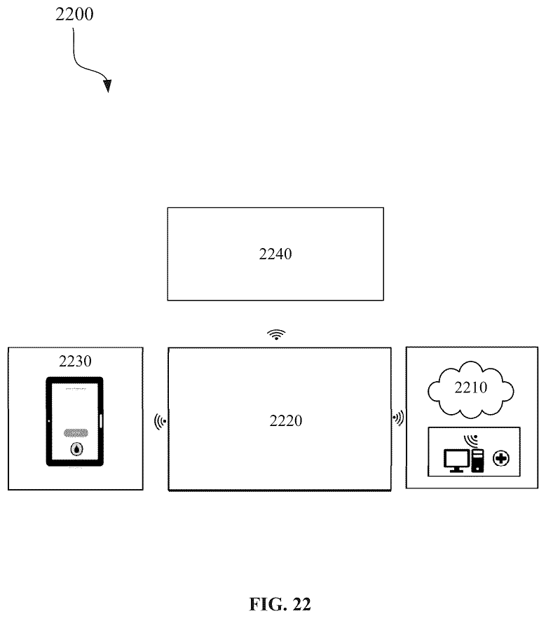

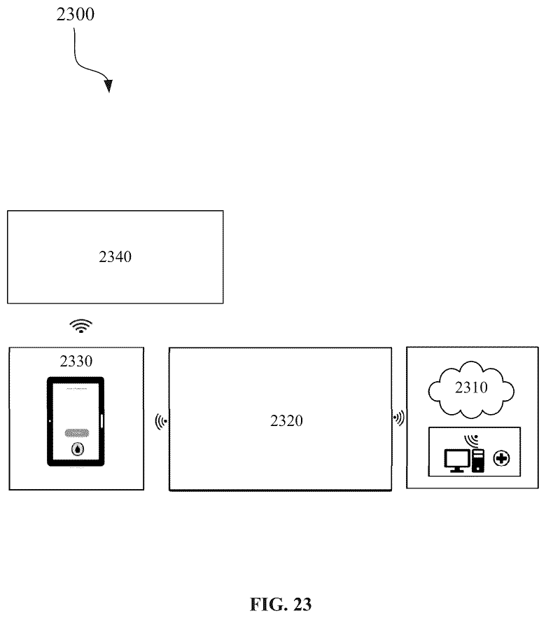

Any of the embodiments in the present disclosure may benefit from the incorporation of additional elements such as elements and systems described in “U.S. PCT Application No. PCT/US2021/038850 which is incorporated by reference in its entirety. For example, the addition of accelerometers may provide positional data to shunt flow measurements or user instructions, wherein positionality of the shunt is known to affect the flow of fluid through the shunt. Other mechanisms of supplying power to a sensor and wireless communication may benefit the present disclosure without departing from the scope of the disclosure. Similarly, the present disclosure may be incorporated as part of a system, for example as shown in FIG. 22 and/or FIG. 23, wherein data is transmitted from a sensor control device to another computing device and/or a remote datastore for later processing, such as a cloud-based data architecture. Such data may then be further stored or provided to further computing devices for display of processed information, such as data over time or alerts, to a user.

Referring now to FIG. 22, a system 2200 may comprise a sensor 2240, such as a thermal flow sensor, in communication with a central communication module 2220. In some embodiments, data, including computer executable instructions, may be transferred between sensor 2240 and communication module 2220. In further embodiments, data may also be stored in a memory storage module within sensor 2240 or communication module 2220 or both. Communication module 2220 may be configured to further transfer data to or from user interface device 2230 and/or remote data store 2210. In some embodiments, user interface device 2230 may comprise a graphical user interface (GUI) to provide information to a user and/or receive inputs from a user. Remote data store 2210 may be configured to provide data access to any number of further systems for data transfer. For example, data may be transferred from remote data store 2210 to another computing system, such as electronic medical record system. In some embodiments, one or more of sensor 2240, communication module 2220, user interface device 2230 and remote data store 2210 may comprise, or communicate with, a memory comprising instructions to execute any number of processes, such as calculations or logic processes based on data transmitted. Communication may be any one or more of known electronic communication methods, such as wired communications, wireless communications, Bluetooth, Zigbee, Wi-Fi, RF, Cellular, LTE, satellite, and the like. For example, sensor 2240 may communicate with communication module 2220 via Bluetooth Low Energy, while communication module 2220 may communicate with remote data store 2210 via Wi-Fi or Cellular methods. It will be appreciated by those skilled in the art that the components of system 2200 could be in further communication with any number of other systems or system components to transmit data between system components.

Referring now to FIG. 23, a system 2300 may consist of a sensor 2340, such as a thermal flow sensor, in communication with a sensor control device 2330. In some embodiments, data, including computer executable instructions, may be transferred between sensor 2330 and sensor control device 2330. In some embodiments, data may also be stored in a memory storage module within sensor 2340, sensor control device 2330, or both. Sensor control device 2330 may be configured to transfer data to or from communication module 2320. In some embodiments, sensor control device 2330 may comprise a graphical user interface to provide information to a user and/or receive inputs from a user. In some embodiments, communication module 2320 may be configured to transmit data to or from remote data store 2310. Remote data store 2310 may be configured to provide data access to any number of further systems for data transfer. For example, data may be transferred from remote data store 2310 to another computing system, such as electronic medical record system. In some embodiments, one or more of sensor 2340, communication module 2320, sensor control device 2330 and remote data store 2310 may comprise, or communicate with, a memory comprising instructions to execute any number of processes, such as calculations or logic processes based on data transmitted. Communication may be any one or more of known electronic communication methods, such as wired communications, wireless communications, Bluetooth, Zigbee, Wi-Fi, RF, Cellular, LTE, satellite, and the like. For example, sensor 2340 may communicate with sensor control device 2330 via Bluetooth Low Energy, while communication module 2320 may communicate with remote data store 2310 via Wi-Fi or Cellular methods. In some embodiments, communication module 2320 may be a part of sensor control device 2330, for example as a cellular or Wi-Fi communications module within a smartphone or other computing device. It will be appreciated by those skilled in the art that the components of system 2300 could be in further communication with any number of other systems or system components to transmit data between system components.

Embodiments of the present disclosure may be implemented through any suitable combination of hardware, software, and/or firmware. Modules and components of the present disclosure may be implemented with programmable instructions implemented by a hardware processor. In some embodiments, a non-transitory computer-readable storage medium including instructions is also provided, and the instructions may be executed by a processor device for performing the above-described steps and methods. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM or any other flash memory, NVRAM, a cache, a register, any other memory chip or cartridge, and networked versions of the same. The device may include one or more processors (CPUs), an input/output interface, a network interface, and/or a memory. Examples of networks include private and public networks, including intranets, local area networks, and wide area networks (including the Internet). Such networks may include any combination of wired and wireless networks and support associated communication protocols.

It will be appreciated that the embodiments of the present disclosure are not limited to the exact construction that has been described above and illustrated in the accompanying drawings, and that various modifications and changes may be made without departing from the scope thereof. The present disclosure has been described in connection with various embodiments, other embodiments of the invention will be apparent to those skilled in the art from consideration of the specification and practice of the invention disclosed herein. It is intended that the specification and examples be considered as exemplary only, with a true scope and spirit of the invention being indicated by the following claims.

The descriptions above are intended to be illustrative, not limiting. Thus, it will be apparent to one skilled in the art that modifications may be made as described without departing from the scope of the claims set out below.

In the foregoing specification, embodiments have been described with reference to numerous specific details that may vary from embodiment to embodiment. Certain adaptations and modifications of the described embodiments may be made. Other embodiments may be apparent to those skilled in the art from consideration of the specification and practice of the embodiments disclosed herein. It is intended that the specification and examples be considered as exemplary only, with a true scope and spirit of the invention being indicated by the following claims. It is also intended that the sequence of steps shown in figures are only for illustrative purposes and are not intended to be limited to any particular sequence of steps. As such, those skilled in the art may appreciate that these steps may be performed in a different order while implementing the same method.

It is appreciated that the above-described embodiments may be implemented by hardware, or software (program codes), or a combination of hardware and software. If implemented by software, it may be stored in the above-described non-transitory computer-readable media. The software, when executed by the processor may perform the disclosed methods. The computing units and other functional units described in this disclosure may be implemented by hardware, or software, or a combination of hardware and software. One of ordinary skill in the art will also understand that the above-described servers may be combined as one server, and each of the above-described servers may be further divided into a plurality of sub-servers.

What is claimed is:

1. A wireless, flexible thermal sensor mountable on a body, the sensor comprising:
a substrate;
a power source;
a thermal actuator supported by the substrate and configured to receive power from a power source and supply thermal energy to a portion of a skin surface of the body;
a temperature sensor supported by the substrate and configured to detect a change in a temperature related to the thermal actuator;
a comparator supported by the substrate and configured to:
receive an electrical signal corresponding to the temperature of the temperature sensor;
compare the received signal to a reference signal; and
output a signal based on the comparison; and
a power control element supported by the substrate and configured to:
receive a signal corresponding to the output signal of the comparator; and
alter the delivery of power to the thermal actuator based at least in part on the signal received from the comparator.

2. The sensor of claim 1, wherein the comparator comprises an operational amplifier.

3. The sensor of claim 1, wherein the reference signal is a voltage differential across a resistor.

4. The sensor of claim 1, wherein the power control element comprises a transistor, a switch, or a variable resistor.

5. The sensor of claim 1, where the thermal actuator comprises an array of resistive elements.

6. The sensor of claim 1, further comprising at least one additional power control element.

7. The sensor of claim 1, further comprising at least a second temperature sensor and a third temperature sensor, wherein the second temperature sensor is positioned on an opposing side of the thermal actuator as the third temperature sensor.

8. A wireless, flexible thermal sensor mountable on a body, the sensor comprising:
a substrate;
a thermal sensor supported by the substrate;
a microprocessor supported by the substrate;
a first region comprising the region of the substrate supporting the thermal sensor;
a second region comprising the region of the substrate supporting the microprocessor;
an adhesive structure, comprising:
a first face configured for bonding to skin;
a second face opposite the first face, the second face comprising:
an adhesive region bonded to the first region of the substrate; and
a non-adhesive region.

9. The sensor of claim 8, wherein the adhesive structure is a multilayer laminate.

10. The sensor of claim 8, wherein the non-adhesive region of the second face of the adhesive structure at least partially overlaps a projected area of the second region of the substrate.

11. The sensor of claim 8, further comprising an enclosure, wherein the enclosure is taller in the second region than in the first region.

12. A wireless, flexible thermal sensor mountable on a body, the sensor comprising:
a substrate;
a power source;
a thermal actuator supported by the substrate and configured to receive power from a power source and supply thermal energy to a portion of a skin surface of the body;
a temperature sensor supported by the substrate and configured to detect a change in a temperature related to the thermal actuator; and
an enclosure comprising a plurality of indicators visible on an external surface, wherein at least two indicators are located on opposite sides of the thermal actuator.

13. The sensor of claim 12, wherein the indicators comprise a raised surface in the enclosure.

14. The sensor of claim 12, wherein the indicators comprise a lowered surface in the enclosure.

15. The sensor of claim 12, where the indicators comprise differently colored regions relative to other regions of the enclosure.

16. The sensor of claim 12, wherein the indicators comprise a change in a contour of an edge of the enclosure.

17. A wireless, flexible thermal sensor mountable on a body, the sensor comprising:
a substrate;
a power source;
a thermal actuator supported by the substrate and configured to receive power from a power source and supply thermal energy to a portion of a skin surface of the body;
a temperature sensor supported by the substrate and configured to detect a change in a temperature related to the thermal actuator; and
an enclosure bonded to the substrate, wherein the external surfaces of the enclosure and the substrate form an unbroken seal around the temperature sensor.

18. The sensor of claim 17, further comprising a conductive coil configured to receive electromagnetic energy for charging a battery.

19. The sensor of claim 17, wherein the substrate comprises an adhesive laminate on at least a portion of one face.

20. A computer-implemented system for monitoring a subdermal fluid conduit, the system comprising:
a wireless subdermal fluid flow sensor configured for attachment to skin;
a sensor control device in wireless communication with the fluid flow sensor, the sensor control device comprising:
a graphical display;
a memory storing instructions;
a processor configured to execute the instructions to:
display a set of instructions to a user to place the fluid flow sensor over the subdermal fluid conduit; and
display a result at least partially based on the received data on the graphical display.

21. The system of claim 20, wherein the set of instructions displayed to the user include instructions to place the sensor on an area of skin such that visible indicators on the sensor align with a subdermal conduit.

22. The system of claim 20, wherein the processor executes additional instructions to determine that a measurement may proceed.

23. The system of claim 20, further comprising a remote datastore, wherein the remote datastore receives information comprising data corresponding to measurements of the subdermal conduit.

24. A thermal flow-sensor mountable on a body, the sensor comprising:
a substrate;
a power source;
a thermal actuator supported by the substrate and configured to receive power from the power source and supply thermal energy to a portion of a skin surface of the body;
a first temperature sensor supported by the substrate and configured to detect a change in a temperature of the thermal actuator;
a second temperature sensor supported by the substrate and configured to detect a change in a temperature associated with a bodily fluid under the skin surface; and
a plurality of current-regulating mechanisms configured to regulate electrical power to the thermal actuator based on the change in the temperature.

25. The sensor of claim 24, wherein the second temperature sensor comprises a plurality of temperature sensors located on opposing sides of the thermal actuator and along a direction of a flow of the bodily fluid.

26. The sensor of claim 24, wherein the bodily fluid comprises cerebrospinal fluid.
